SEQUENCE LISTING (4 sequences)

SEQ ID NO: 1 - DNA, 12878 bp, Artificial Sequence
  Homo sapiens stanniocalcin 1 (STC1) gene sequence
agtttgcaaaagccagaggtgcaagaagcagcgactgcagcagcagcagcagcagcggcggtggcagcagcagcagcagcggcggcagcagcagcagcagcggaggcaccggtggcagcagcagcatcaccagcaacaacaacaaaaaaaaatcctcatcaaatcctcacctaagctttcagtgtatccagatccacatcttcactcaagccaggagagggaaagaggaaaggggggcaggaaaaaaaaaaaacccaacaacttagcggaaacttctcagagaatgctccaaaactcagcagtgcttctggtgctggtgatcagtgcttctgcaacccatgaggcggagcagaatgactctgtgagccccaggaaatcccgagtggcggctcaaaactcaggtaagcagcaaacccaagagcggtttctcccccaaagagctgtcctcatttgcctctcccttttgcaactgtgtctgtgacggctgatcttgataataaatgtgcttcatgcctgatggcaataaactgccagtgtaatccaatagccttaggcagtggagctcctttgtttaataaattgcatgcaactaaacgaagaagctggacgctctgctgagggtatttcattgcataagcctagcctgattgcctgaaatctggcacgtaccctcttggagggggaggggtgagaggggaggaaaggcttgaatgttggcatgcttcaaagcgctctctatactttccagaagctgatctaaggtaagacctggcttgtttgatgctgtccctcttcctttcctcttaagactctacctcttcacttcttgacttctctacctaagagaatacacagaaaagctccctcttcagactgattttcagaaccatagccctcaagtctgacaaatccagggcggattcagagaaaaccctatacatcccacccccaaccctgctgcctgtctccctcctggctctgtagggctccattcagtcgtccctgcagcccgtagccagagagcccctagaaaaaaaaaattgtgaaacgtagcagctgtttctccaagggtaaggtctcactttcgtcttgactcttggagcagttaattgtgcattatcttggcttctgaaggaaaagaatcattaggaagcctgcattgacacctctgcctcttgacttctctggctccacttgttccccttccaatactgccctctgtcctccaactgtcagcaaagaccggatcacaggtgtcaaaatgctgcagatgatgatccccttggaagagcacaggtccctgccgagaggagggcttttgcttcacttccttttgtacttagttgctatagagatgcagtgattcacaattcgagcttgcctaaaacaatagcagccctaatgcatattaaaactgcttaaagcaaagttataatttaaacccgtggaaatatataattagggaaatgacttgtaacatcctagaggctgcgggtggtgggagggggtggggaagactaactgaaccatgcttttgatctcagaggagaatcctatggcatgaaggctgcagatcattccttgcctagctgcctgccagaacaccccccttctgtgggagaaaaattacttttatgggagatcttatccagaatgtagaaagcctcatgcaagttttttgcggttttcttccagtcttgcatagggaggccttggcctggttattccacacctgtgtttcttctttctttctttctttctttctttcttttttaaaaggcttttaatatgaaagagattatgcatacctctaagcactgtcctagtggagggttggggtgaaacatgaacggatcagatttctttttgccaatatgacggtaagttaaaagcctgaaatcagaagggtctccaagtggactcctggcacagtcttctctgccatttagactaaaatcccatgctttttactcgggagtgcatttttccaaagctagatggattccctggcccttttacctcttaaacctgaccactagccactgtctccttccttcccatgctttcaagtgctaagcaatgacctttgattctatggaatggcttaaaggcactgtgcatttccagacttgagcctcacttctccagacaccaaaattgtaggtaatggtgaaacagggagcgtgtgtctgaaacagagaactccctccctgtccatcatgtactccagcaatcataaacaaaaggagcctgtctatcctctctttctcaatcaaatgggtctatcaattgcccctcatgacagattcctaggataagttcttctgacctcatgtccttgaaattcacctctgcacccacttgatcccacccagccattcctcactgtttgacccagaatggatggatttgtctctttcagctgaagtggttcgttgcctcaacagtgctctacaggtcggctgcggggcttttgcatgcctggaaaactccacctgtgacacagatgggatgtatgacatctgtaaatccttcttgtacagcgctgctaaatttgacactcaggtaataaaaccttgacccctgctcccactggctgcttgtctttaacagtgtgttagaccatggttcttaggaaccagtcttgtagggaaccagccctgcttctcagcttccccactctaaattcccatctcagcatgtgtatgcacaagtgtgtgcatgcacacacacacacacacacacacacagaggcactcatctggccaggaaataggttctgtataagacacgccccactcactctaatgcatgattctaaggagctagtagaggctgtcttataaggccaggggtaataagccaaaaagattttcctctgtaagaggacataaaaacgtgcatagagcaaggaagttctctctgcatgacctagcatactagcttttgatctgaattctcaaagtctcaatctcaagaatatgaagtcagattacgactatttttaagctgcagaatggttgatcacggttcatcttttttttctgtaatttcatttcggtagagcttaattctcaagggaatccagggagatggggagagggcttttttttttttcaatccttagccgagacatgcaatcttctcattgcaaggatatgcagatggccatgatacgttgcccagatgagcccccttaaattcctccatgtatatgcccatcctctttgtcaggtcaagctaaagacctccctcttgtccttcctccatggcagggaaaagcattcgtcaaagagagcttaaaatgcatcgccaacggggtcacctccaaggtcttcctcgccattcggaggtgctccactttccaaaggatgattgctgaggtgcaggaagagtgctacagcaagctgaatgtgtgcagcatcgccaagcggaaccctgaagccatcactgaggtcgtccagctgcccaatcacttctccaacaggtacaaactgagtctactttttcacaatcaggggactgggagcaggtggctggctggcagagccagactgggagggagggttaaattgccacagtcatgctcttattttagcttgcagtcttcccagcttttgcaggaaaaaaatatgtttgtgattggttgtgacagtttaagcagcttgggcttttttttccctactgttataggaaaagtcttttcatgcagctcatagaaagccagcctcatctgcagcatccctgggtgctttcattgaccattttttgccataagcctgaagggaatggtgaagtttctggaaacaagcttttaagacatcagatttggctgaaaccatgaccgtgcaacaacacaattggccatgattaaaaaaaaaaaaaaaaaaaaaagccctctggttaaatgtcataatgcagattttaagtctccatctatttcaaatcagtcccaaacacagtcagagatttcactgggtagcccagagcaagccatctcacttccctggggctcagatttcctttgtaaaaagcaaaatggctggactggaccattttcaggtttccttccaggccttatgttatcccattcctctagcccaagatttcctagaatgagtgtgttttatggctgtccagatcccattctcatcttagtattcaaacaccacccaaggccactgagctagtacagtaacatctgggcattgtacttttcagtgtttttaccagccaggagtgaattcctgttacatgagacagctggcatcctcagttcatgtgcgtggcctggaaaggttcccagcaagaactaagagatgccaatggggttggcgtggaggtctggcaggggcagtgcagaccggtcaagtttagccccctgtgatatccctcagcctaaaaacttgactcccagccttgaatttctatgattctttgactctccactcaaatctgcacatatccacaatgatcagtgctttgcatgttactctgaccacctgagagttatcacaggcttatctgattaatttcctagccaggtgccaccacccttcttaagacagtggcaacagccaagtccctctggggcttacaagtgatttagtcatgtacatacatgcaacaaaaaagtagttcctccttagggaaaggctaaggtgcctaacacaaacgctgtgtaaaatttctttttcacccttaactacaggtgagtgcccttcagaaccacaaatcaatttgtaaaaatcacttctctagtccttggaagtgactttatcatggggttctaaatggtttcctcagaacagaaaaatccaaccattttctcaagtgggctgtctctatataactcttggttttaaatttcccctaggaagattcttattgcacagaggaaacaggtgcagagggtggggaggggagatctgattaattattggagacttccttcaccattaggaaaagaaatgaagaattcttagccagacaggggctaaggacaaagtatgtgtttgtgtggtagttaggggtggggggagaggtacattagcaaagccactattacaatttagatccttaaggattctgtgacaaatcatgatctataatacggcatatttccaattttgaggcgatactaatctccctatttcatctgaagtagcatcttccattagaaaggcagagaggtctttaggcagaactatatagctactcgactagaggtcccaatttatctaagaagagactagtaaatttttaaccaggctattatttcttctaatcagtgccttattttcaacacctttagaaaatggtcacaaagaaagtcttcttatctccctctttcatcttagggactcattaggaaaggtcaattggagaaaaaaaaaatgtggccagcccttgaaaaccggaatcttccatgcaacctttctccaaaaatttcactttaacaggctatattattcctgctcaggattgtagaaatgtccggttccatgctgggcttgctttgctccattgaagctgctcaccttcctgagacatcctgctttcccagcctctatcccaccatagacacacgctaagtcagtgccaattggcacagaattgcaagaggaagtagttacaggttcattccaccttcctgtgacttatctagcatgagatttggggctgggagaatccaaaagcctgtgactggaccaggagccagaacaaggggattttccatgacagaaatgcacctagagggtaaagaggtagctggagtttccacctgcctgaatttgcagactatgctttcaaccctgtgggaagcaggcaaaattgatcaggccagcagggcggtgcgaggcttgggaaccacttagaccaacccagatccagagtgggaaaactgacaaagcttccaactctctagggcatggatagggaacaggaaacagaagaataatattgatcaagggaaacaaaggactatttataaaccctcacacagaacgttcctaggcagtatgtgtgtatttgggtaagcatggctgaaacagtagctatggagaatatctgatcttattctttatcaaaaaaaaaaaggtcagggatggaattttcaaagtgagaaaaaagaaacataattaaaaaggtcctttcgggctgggtgatcagaacacttagtccaccaagtctttccaacacatctcctccatcctccataaagataaagaaaggaatcttcttcttctttttttttttttttttttttttttttgacagagtatcgctctgttgcccaggctggagtacagtggtgagatctcagttcactgcaacctccatctcctgggttcaagcaattctcctgcctcagcctccagagtagctgggattacaagcactcaccaccacgcccagctaatttttgtatttttagtagagacagggtttcatcatgttggccgggctggtcttgaactcctgacctcaggtgatccacccatcttggcctcccaaagtgctgggactgcaggcatgagccaccatgcccagccaagaaaggaatcttcttaagcagtcctaactaccactgcaaaggaggggatgtaagcagagctgctctattttttgtaaaattgagaagaaaaatgtgtattgaatccctagaaacatagaatgtcatagttggagatgactgctgaggttaactagactaactgcctcattttactgattaaagaataaaggctcagagagtggagtgacctgctgaaggtcacacagcttgtgaataactaagttgggcttagaactcatgtggccttgagccaagctatttgatttctacttcaaaaatggcagccagcaaaatctgcaggaaatagccacaacccaaaaatagaataccaagcacaattgaatatatgtgcaccgtggtagtccacaattaaatttgcgtcctttctctcccaaaaggatggacacatcctaccaatagctataggaactataagaccatagcacaagaaggtgctcactaaataggtgacgattgctaagtcaactcttgctggagacacaacctcttaaataatccatctcacttagtgacacaggaaagggctatgtcattggcaagcatgatcccagaggtaaatggctattccagcccttactggacaggcactggttaacctattgctcatactgagaaactggtgacatcaaataatcctgacaaattttccagcctttgaagaactactaatggagacatggactttgttttatattctaaagagctttctcagcctgcccttgctgagttaacaaaagccccatgagggaaatagggtagattccagtgggggtgggtggttctcactaagagccctcaggctggcaagggtcactgtcaagaaacaccaaggtcaggtgtcatatctgagccagcagacaaagcaacagtaaaggtagaaaggtagaaaccctccaaaaaagagcaggcactctgacaaagtggggcctgaaggaaatgtatggccctgaagctgcaaagctggctgcagaagcttaattaccttaagggtactagacttctttgtccagacaagagctttcaagtccttctaaggaagaactagcatttctttttatccctttctagggaaagattgtattttccagcctatctggggactcatctggcatgaacagctctttctagaaatggaagggttaacttctaagttataaagtccttattggagggatgggagatcagaaaccacccaccaggatctaatcaccctctgaaatagacacagccatccaaagacccaggtcctagcagagcccaagagccaggtgatgcctggagatctatatgggtgtgtagatgtcagaagctgctggtgcaaagttgtttattctatgtttctccaaaactggttttctcctcatttccccatccaaaggagatctcctttgactcatgtgctttgtaattccaaagagttggacaatgttcctctgtgccagagagtcccagttttgaccacagctggatataattctccctggagggaagggtttcaccaggcctgtgtcagcaaccaagacactgcacttgcctctcagtccccagcttccttctgctgtgccacagaagggtgaccatatcacctcattacccacgttccatgctatttaccacacatctttgaaataacctctctgggggattaagttattctagaaagatgtttgatcctgtgcactgtttggtgagttaaaaataaacaggtttcttttcacggaagacctggtggccccagagatccttgggctcctctgagaaaaagttctctccatgtaaatactgcaggtcactgtcactgagtctcttattagcgaacacagaggtaatgggttttgacaactcaactgcgactacaaaatggaccaaatgcatcccagtctaaattggataatacctgaactgtcttcccaaataacaggacaagtcattgatatgccctaaatattagacagtttaatcctttctgagccacaaagcttagctacattgcttgggggtatttaaagaattcgctgcaaagatgaaggtaactgataccaaacacaaggggttttggtaaaatttactaaaactacagataagttttcttctgggaatcactacttattggagagaattatattcttaaaactccatttccttagtggctgcaaaataaatcaacaaatgtgcagtgatcaggcaatgtagcacgatggcaccgggccataaggaatacaaagaaggacacaaatcctgccttcagtaagcttacagtctagtcagcgaaagaaagccatcacaaaaccatcaaatgctggcctatgtgatggcaaacaaagatctataaatatccaaaaagggaagaggttagcaaactctggtatagtagaagaaggattcatgaattatagtagcatgaacccttaaaaaccaccaatatttccatttaaatagacttcttgcctaggatttggccttctgactacaatatacctttgaaggagggctgaagatggagtcagtcaatatcttttatcagggctgtccaagtatgtgataagggcattagattttgtagaaaaatgcatggcacacagaagagggggccttagacagaagtttcatcagaagatgcacagatctgcatacacacacacacacacacacctgtgcccacacaccaccaaaaaaaggaaaattacaaaaaagcatcaaaataattacacatgcatgtacaatggctactaatctcagtgatgagcattggactcacttcattctaatttgcttcacctcatccaccttagtttcttctctacaaaatgagagttgaactagtcaccaaatggtctctgtggtcccagctagctctgtaattctgtttctaaaaatgtcagtcaccacagtcagcctgctcttcaaagtgtgatctaaatctcctcaactctaaacatgaagatctgtcttgcattgtgtggtaaaccctaggagcagatgcattgaaactagggtgacatggcacaagggttagagagcaaaagccaccttcagctgatgaactattgatgtttgcatggtcaggccattagggactcgtgttggctagagcccgggtgaaagcctgggtctcgccgggctctcactgggtgaccaccatatgcactctctttcttgatgcagatactataacagacttgtccgaagcctgctggaatgtgatgaagacacagtcagcacaatcagagacagcctgatggagaaaattgggcctaacatggccagcctcttccacatcctgcagacagaccactgtgcccaaacacacccacgagctgacttcaacaggagacgcaccaatgagccgcagaagctgaaagtcctcctcaggaacctccgaggtgaggaggactctccctcccacatcaaacgcacatcccatgagagtgcataaccagggagaggttattcacaacctcaccaaactagtatcattttaggggtgttgacacaccagttttgagtgtactgtgcctggtttgatttttttaaagtagttcctattttctatcccccttaaagaaaattgcatgaaactaggcttctgtaatcaatatcccaacattctgcaatggcagcattcccaccaacaaaatccatgtgaccattctgcctctcctcaggagaaagtaccctcttttaccaacttcctctgccatgtttttcccctgctcccctgagaccacccccaaacacaaaacattcatgtaactctccagccattgtaatttgaagatgtggatccctttagaacggttgccccagtagagttagctgataaggaaactttatttaaatgcatgtcttaaatgctcataaagatgttaaatggaattcgtgttatgaatctgtgctggccatggacgaatatgaatgtcacatttgaattcttgatctctaatgagctagtgtcttatggtcttgatcctccaatgtctaattttctttccgacacatttaccaaattgcttgagcctggctgtccaaccagactttgagcctgcatcttcttgcatctaatgaaaaacaaaaagctaacatctttacgtactgtaactgctcagagctttaaaagtatctttaacaattgtcttaaaaccagagaatcttaaggtctaactgtggaatataaatagctgaaaactaatgtactgtacataaattccagaggactctgcttaaacaaagcagtatataataactttattgcatatagatttagttttgtaacttagctttatttttcttttcctgggaatggaataactatctcacttccagatatccacataaatgctccttgtggccttttttataactaagggggtagaagtagttttaattcaacatcaaaacttaagatgggcctgtatgagacaggaaaaaccaacaggtttatctgaaggaccccaggtaagatgttaatctcccagcccacctcaacccagaggctactcttgacttagacctatactgaaagatctctgtcacatccaactggaaattccaggaaccaaaaagagcatccctatgggcttggaccacttacagtgtgataaggcctactatacattaggaagtggcagttctttactcgtcccctttcatcggtgcctggtactctggcaaatgatgatggggtgggagactttccattaaatcaatcaggaatgagtcaatcagcctttaggtctttagtccgggggacttggggctgagagagtataaataaccctgggctgtccagccttaatagacttctcttacattttcgtcctgtagcacgctgcctgccaaagtagtcctggcagctggaccatctctgtaggatcgtaaaaaaatagaaaaaaagaaaaaaaaaagaaagaaagagggaaaaagagctggtggtttgatcatttctgccatgatgtttacaagatggcgaccaccaaagtcaaacgactaacctatctatgaacaacagtagtttctcagggtcactgtccttgaacccaacagtcccttatgagcgtcactgcccaccaaaggtcaatgtcaagagaggaagagagggaggaggggtaggactgcaggggccactccaaactcgcttaggtagaaactattggtgcttgactctcactaggctaaactcaagatttgaccaaatcgagtgatagggatcctggtgggaggagagagggcacatctccagaaaaatgaaaagcaatacaactttaccataaagcctttaaaaccagtaacgtgctgctcaaggaccaagagcaattgcagcagacccagcagcagcagcagcagcacaaacattgctgcctttgtccccacacagcctctaagcgtgctgacatcagattgttaagggcatttttatactcagaactgtcccatccccaggtccccaaacttatggacactgccttagcctcttggaaatcaggtagaccatattctaagttagactcttcccctccctcccacacttcccacccccaggcaaggctgacttctctgaatcagaaaagctattaaagtttgtgtgttgtgtccattttgcaaacccaactaagccaggaccccaatgcgacaagtagttcatgagtattcctagcaaatttctctctttcttcagttcagtagatttccttttttcttttctttttttttttttttttttttggctgtgacctcttcaaaccgtggtacccccccttttctccccacgatgatatctatatatgtatctacaatacatatatctacacatacagaaagaagcagttctcacaatgttgctagttttttgcttctctttcccccaccctactccctccaattcccccttaaacttccaaagcttcgtcttgtgtttgctgcagagtgattcgggggctgacctagaccagtttgcatgattcttctcttgtgatttggttgcactttagacatttttgtgccattatatttgcattatgtatttataatttaaatgatatttaggtttttggctgagtactggaataaacagtgagcatatctggtatatgtcattatttattgttaaattacatttttaagctccatgtgcatataaaggttatgaaacatatcatggtaatgacagatgcaagttattttatttgcttatttttataattaaagatgccatagcataatatgaagcctttggtgaattccttctaagataaaaataataataaagtgttacgtttta

SEQ ID NO: 2 - protein, 247 aa, Artificial Sequence
  Homo sapiens stanniocalcin 1 (STC1) amino acid sequence
MLQNSAVLLVLVISASATHEAEQNDSVSPRKSRVAAQNSAEVVRCLNSALQVGCGAFACLENSTCDTDGMYDICKSFLYSAAKFDTQGKAFVKESLKCIANGVTSKVFLAIRRCSTFQRMIAEVQEECYSKLNVCSIAKRNPEAITEVVQLPNHFSNRYYNRLVRSLLECDEDTVSTIRDSLMEKIGPNMASLFHILQTDHCAQTHPRADFNRRRTNEPQKLKVLLRNLRGEEDSPSHIKRTSHESA

SEQ ID NO: 3 - DNA, 21 bp, Artificial Sequence
  STC1_qRT PCR primer F
agcgctgctaaatttgacact

SEQ ID NO: 4 - DNA, 23 bp, Artificial Sequence
  STC1_qRT PCR primer R
ctttggaaagtggagcacctccg

(19) United States
(12) Patent Application Publication
(10) Pub. No.: US 2024/0301503 A1
(43) Pub. Date: Sep. 12, 2024

(54) NOVEL BIOMARKER FOR DIAGNOSING AND PREDICTING METASTASIS OR PROGNOSIS OF VARIOUS CANCERS, AND USE

(71) Applicant: Dong-A University Research Foundation for Industry-Academy Cooperation, Busan (KR)

(72) Inventors: Sun-Hee LEEM, Busan (KR); Jeong-Yeon MUN, Busan (KR); Mi-So JEONG, Busan (KR); Min-Hye KIM, Gimhae-si (KR); Gi-Eun YANG, Busan (KR)

(73) Assignee: Dong-A University Research Foundation for Industry-Academy Cooperation, Busan (KR)

(21) Appl. No.: 18/573,338

(22) PCT Filed: Jun. 22, 2022
(86) PCT No.: PCT/KR2022/008902

§ 371 Date: Dec. 21, 2023

(30) Foreign Application Priority Data
Jun. 22, 2021 (KR) 10-2021-0080807
Jun. 22, 2022 (KR) 10-2022-0076394

(51) Int. Cl. C12Q 1/6886; C12Q 1/6809

(52) U.S. Cl. CPC C12Q 1/6886; C12Q 1/6809; C12Q 2600/112; C12Q 2600/118; C12Q 2600/136; C12Q 2600/158; G01N 2333/00; G01N 2333/575

(57) ABSTRACT

The present invention relates to a novel biomarker for diagnosing cancer, and a use thereof. STC1, a novel biomarker for diagnosing or predicting the prognosis of cancer according to the present invention, was found to be related to poor prognosis of cancer patients according to the expression level, and was found to be overexpressed in various cancer cell lines. In addition, STC1 was found to be a biomarker related to the proliferation, invasion, and migration (metastasis) of cancer cells. In addition, it was found that STC1 is detected in the serum or urine of bladder cancer patients, and can be effectively used for diagnosing and predicting the prognosis of bladder cancer by identifying differences in expression according to the patient's clinical stage.

### TECHNICAL FIELD

The present invention relates to a novel biomarker for diagnosing and predicting the metastasis or prognosis of various cancers and a use thereof.

### BACKGROUND ART

Cancer refers to the abnormal growth of cells, and refers to a malignant tumor in which cells lose their normal regulatory mechanisms to continuously proliferate, infiltrate nearby tissues, migrate to distant parts of the body, or promote new vascular growth through which the cells receive nutrients. Cancerous tissues (malignant tumors) may be classified into tumors (leukemia and lymphoma) in blood and hematopoietic tissues and “solid” tumors (solid cell mass), which are usually referred to as cancer. Types of cancerous solid tumors include carcinomas or sarcomas, and specific cancers are further classified by first occurring organs and occurring cell types. Leukemia and lymphoma are cancers of blood, hematopoietic tissue, and immune system cells, and the leukemia develops in hematopoietic cells and inhibits the generation of normal blood cells in the bone marrow. Cancer cells in lymphoma enlarge lymph nodes and form large masses in the armpits, groin, abdomen, or chest. Carcinoma is cancer that occurs in the inner cells of the skin, lungs, digestive tract, and internal organs, and examples of carcinoma include cancer that occurs in the skin, lungs, colon, gastric, breast, prostate, and thyroid gland. The sarcoma is cancer of mesodermal cells. The mesodermal cells generally form muscles, blood vessels, bones, and connective tissue, and examples of sarcomas include leiomyosarcoma (cancer of the smooth muscles in the walls of the digestive organ), osteosarcoma (bone cancer), and the like.

Generally, in the early stages of cancer, tumor formation may be confirmed by performing X-ray, ultrasonography, or computed tomography on suspected patients, but whether the confirmed tumor is cancer is determined by additional diagnosis. To specifically diagnose cancer, tumor tissue is collected through biopsy or surgery, and samples from suspected areas are examined under a microscope to identify cancer cells. In addition, when cancer is suspected as a result of examination or imaging, additional evidence for cancer diagnosis may be obtained by measuring the levels of tumor markers (substances secreted from specific tumors into the bloodstream), and for people diagnosed with specific cancers, tumor markers may be effectively used to monitor the therapeutic effect and detect the possibility of cancer recurrence. In some cancers, tumor marker levels decrease after treatment, and then increase again when the cancer recurs, and some tumor markers are able to be detected in biological samples, including blood, but there are also markers that are detectable only in tumor tissue. These tumor markers are commonly called biomarkers.

Meanwhile, bladder cancer is the most common cancer among urinary system cancers, and as society gradually continues to age, the number of patients diagnosed with bladder cancer is increasing. When patients with bladder cancer are diagnosed, approximately 70% of patients are diagnosed with superficial bladder cancer that has not invaded the muscle layer of the bladder, and the 5-year survival rate when treated reaches 70%. However, 50% or more of recurrence not only appears as superficial bladder cancer, but some patients also develop invasive or metastatic bladder cancer that has invaded the muscle layer. The biggest problem in successful treatment of bladder cancer patients is anticancer drug resistance and frequent recurrence. For this reason, patients with bladder cancer need constant monitoring, and they often undergo cystoscopy after the examination, which is expensive and has side effects. In addition, despite treatments such as chemotherapy, recurrence problems in bladder cancer patients continue to reduce the quality of life and cause financial distress. Therefore, it is required to develop a biomarker that makes bladder cancer diagnosis safer and easier and can even predict anticancer drug resistance and prognosis of the patients.

### DISCLOSURE

### Technical Problem

An object of the present invention is to provide a novel Stanniocalcin-1 (STC1) biomarker for diagnosing or predicting the metastasis or prognosis of cancer.

Another object of the present invention is to provide a biomarker composition for diagnosing or predicting the metastasis or prognosis of cancer, including an agent capable of measuring the expression level of Stanniocalcin-1 (STC1).

Yet another object of the present invention is to provide a kit for diagnosing or predicting the metastasis or prognosis of cancer including the composition.

Yet another object of the present invention is to provide an information providing method for diagnosing or predicting the metastasis or prognosis of cancer including: isolating a biological sample from a subject:
measuring the expression level of Stanniocalcin-1 (STC1) in the isolated biological sample; and
comparing the expression level of STC1 with a reference value of a control group.

Yet another object of the present invention is to provide a screening method of an anticancer agent including: isolating a biological sample from a subject;
treating the isolated biological sample with a candidate substance;
measuring the expression level of Stanniocalcin-1 (STC1) in the biological sample treated with the candidate substance; and
comparing the expression level of STC1 with a reference value of a control group.

Yet another object of the present invention is to provide a method for diagnosing or predicting the metastasis or prognosis of cancer including: isolating a biological sample from a subject;
measuring the expression level of Stanniocalcin-1 (STC1) in the isolated biological sample; and
comparing the expression level of STC1 with a reference value of a control group.

### Technical Solution

In order to achieve the object of the present invention, an aspect of the present invention provides a novel Stanniocalcin-1 (STC1) biomarker for diagnosing or predicting the metastasis or prognosis of cancer.

Another aspect of the present invention provides a biomarker composition for diagnosing or predicting the metastasis or prognosis of cancer, including an agent capable of measuring the expression level of Stanniocalcin-1 (STC1).

Yet another aspect of the present invention provides a kit for diagnosing or predicting the metastasis or prognosis of cancer including the composition.

Yet another aspect of the present invention provides an information providing method for diagnosing or predicting the metastasis or prognosis of cancer including: isolating a biological sample from a subject;
measuring the expression level of Stanniocalcin-1 (STC1) in the isolated biological sample; and
comparing the expression level of STC1 with a reference value of a control group.

Yet another aspect of the present invention provides a screening method of an anticancer agent including: isolating a biological sample from a subject;
treating the isolated biological sample with a candidate substance;
measuring the expression level of Stanniocalcin-1 (STC1) in the biological sample treated with the candidate substance; and
comparing the expression level of STC1 with a reference value of a control group.

Yet another aspect of the present invention provides a method for diagnosing or predicting the metastasis or prognosis of cancer including: isolating a biological sample from a subject;
measuring the expression level of Stanniocalcin-1 (STC1) in the isolated biological sample; and
comparing the expression level of STC1 with a reference value of a control group.

### Advantageous Effects

According to the present invention, STC1, a novel biomarker for diagnosing or predicting the prognosis of cancer, was found to be related to poor prognosis of cancer patients according to the expression level, and was found to be overexpressed in various cancer cell lines. In addition, STC1 was found to be a biomarker related to the proliferation, invasion, and migration (metastasis) of cancer cells. In addition, STC1 has been detected in the serum or urine of patients with bladder cancer, and it can be effectively used in the diagnosis and prognosis prediction of bladder cancer by confirming the differences in expression according to the patient's clinical stage, and can be useful in related industries.

### DESCRIPTION OF DRAWINGS

FIG. 1 illustrates identification and quantitative comparison of conditioned media for T24 (P0) and GRC1-P15 (P15) cells. FIG. 1A is a schematic diagram illustrating a secretome analysis process for identifying secreted proteins from serum-free media of P0 and P15 cells using LC-MS/MS. FIG. 1A is a heatmap illustrating the expression of various secreted proteins for three independent conditioned media of P0 and P15 cells. A right diagram illustrates STC1 protein expression and ponceau staining results in conditioned media derived from P0 and P15 cell cultures. FIG. 1B is a Venn diagram illustrating the number of proteins identified in conditioned media of P0 and P15 cells. FIG. 1B illustrates top 13 standard pathways that changed most significantly in the conditioned media of P15 cells compared to P0 cells. FIG. 1C illustrates total 27 proteins summarized according to locations and overexpression of 386 proteins identified specifically in P15, between conditioned media of P0 and P15 cells.

FIG. 2A and FIG. 2B illustrate results of comparing STC1 expression and expression of secretory STC1 protein in cell lysates and cell culture conditioned media of glioblastoma (U251), lung cancer (A549, H460), colon cancer (LoVo, HCT116), prostate cancer (DU145, PC3), bladder cancer (T24, 5637), breast cancer (MDA-MB231, SKBR3), pancreatic cancer (Miapaca2, CFPAC1), gastric cancer (AGS), and ovarian cancer (SKOV3) cell lines, as compared with human fibroblasts (Nuff).

FIG. 3 illustrates that STC1 is a factor for predicting poor prognosis in bladder cancer. FIG. 3A illustrates a Boxplot of STC1 expression levels in three bladder cancer cohorts. FIG. 3B is a table for describing three bladder cancer cohorts. FIG. 3C illustrates expression profiles of STC1-associated genes. A total of 367 genes in which expression levels were closely related to STC1 were selected for cluster analysis (Pearson correlation test, P<0.001, |r|>0.4). The corresponding patients were divided into two groups, high and low STC1 expression. Progression-free and cancer specific survivals according to STC1 expression were shown in the Korean bladder cancer patient cohort (GSE13507). FIG. 3D illustrates Gene Ontology-based biological functions determined using DAVID software. The thresholds for significance are P<0.001 and FDR<0.25. In addition, the right chart illustrates 21 genes out of a total of 367 genes with expression levels closely related to STC1. FIG. 3E illustrates gene expression profiles of STC1-associated genes. Among 367 genes obtained from the Korean bladder cancer patient cohort (GSE13507), 345 genes common to the Lund cohort were analyzed. Patients were divided into two groups, STC1 high and STC1 low, and a graph shows progression-free and cancer specific survivals according to STC1 expression in the Lund cohort. FIG. 3F illustrates gene expression profiles of STC1-genes, in which among 367 genes obtained from the Korean bladder cancer patient cohort (GSE13507), 308 genes included in common with a Yonsei cohort were analyzed. Patients were divided into two groups, STC1 high and STC1 low, and a graph shows cancer specific survival according to STC1 expression in the Yonsei cohort.

FIG. 4 illustrates the content that the expression of STC1 regulates the growth and proliferation of bladder cancer cells. FIG. 4A and FIG. 4B illustrate STC1 mRNA and protein expression levels via qRT-PCR and Western blot in STC1 overexpressing and knockdown cells, respectively. FIG. 4B illustrates results of cell viabilities of cell lines transfected with STC1 overexpressing vectors (pSTC1) #1, #2, and #3 and STC1 knockdown siRNA (siSTC1) #1, #2, and #3 through MTT assay. FIG. 4C illustrates clonogenic assay results of cells transfected with pSTC1 #1 and siSTC #2, which have the best STC1 overexpression and knockdown efficiency among three candidate groups. Data were expressed as mean+SD from at least three independent experiments. **, P<0.01: ***, P<0.001.

FIG. 5 illustrates that STC1 promotes proliferation, invasion, and migration of bladder cancer cells. FIG. 5A illustrates results of expression levels of STC1 mRNA and protein in control cells and STC1 overexpressing (STC1 OE), i.e. STC1 overexpressing cell line. A left graph of FIG. 5B shows MTT assay results of confirming the cell viability in STC1 overexpressing cells compared to control cells. A right graph of FIG. 5B shows a clonogenic assay result of exhibiting an effect on tumorigenesis of STC1 overexpressing cells compared to control cells. FIG. 5C illustrates results of cell invasion and migration assay using transwells of STC1 overexpressing cells compared to control cells. **, P<0.01: ***, P<0.001.

FIG. 6 illustrates (FIG. 6A) positive correlation association between STC1 and EMT-related genes (11 genes, VIM, ZEB1, ZEB2, SNAI1, TWIST1, TWIST2, MMP1, MMP3, MMP9, NCAD, and CD44) and (FIG. 6B) negative correlation association between STC1 and mesenchymal-epithelial transition (MET)-related genes (3 genes, SDC1, SDC2, and ECAD) in a Korean bladder cancer patient dataset.

FIG. 7 illustrates results that STC1 expression increases the expression of EMT-related genes in bladder cancer cells. FIG. 7A illustrates mRNA levels of EMT and MET-related genes (MMP1, MMP2, MMP9, VIM, SNAIL, SLUG, ZEB1, ZEB2, TWIST, NCAD, SDC1, SDC2, and ECAD) expressed in cells transfected with STC1 overexpressing vector (pSTC1) compared to control cells (pcDNA). FIG. 7B illustrates a result of Western blot assay obtained by comparing the protein expression of EMT and MET-related genes (MMP1, MMP2, MMP9, NCAD, VIM, SNAIL, and ECAD) identified among the genes with control cells (pcDNA). ns, no significant; *, P<0.05: **, P<0.01: ***, P<0.001.

FIG. 8 illustrates that STC1 overexpression promotes tumor growth in vivo. FIG. 8A is a process diagram illustrating a mouse tumor formation experiment process. Subcutaneous injection was performed on day 7 after a mouse environmental adaptation period, and mice were randomly divided into two groups [n=7 per group; Con (control cells), STC1 OE (STC1 overexpressing cells)]. Control and STC1-overexpressing cells (1×10⁶ cells) were injected into the mouse flank and body weight was measured weekly. FIG. 8B illustrates that after the last measurement, mice were sacrificed and tumor tissue was harvested. FIG. 8B illustrates results of measuring tumor volumes with calipers for 35 days. A right diagram shows images of tumors surgically removed from mice. FIG. 8C illustrates staining photograph results of confirming H&E and STC1 expression immunohistochemically in mouse tumor tissue formed by injection of STC1 overexpressing cells. *, P<0.05: **, P<0.01: ***, P<0.001.

FIG. 9 illustrates that STC1 overexpression promotes tumor metastasis to the lung in vivo. FIG. 9A is a process diagram illustrating a mouse tumor metastasis experiment process. Intravenous injection was performed on day 7 after a mouse environmental adaptation period, and mice were randomly divided into two groups [n=7 per group: Con (control cells), STC1 OE (STC1 overexpressing cells)]. Control and STC1-overexpressing cells (5×10⁵ cells) were injected into the mouse tail vein and body weight was measured weekly. FIG. 9B illustrates a result of confirming images and the number of mouse lung tissues in which lung metastases formed by injection of a control and STC1 overexpressing cells were confirmed. A right diagram is a result of confirming H&E, Ki67 and STC1 through immunohistochemistry. FIG. 9C illustrates results of H&E staining and STC1 expression level of bladder cancer tissue of Grades 1, 2, and 3 of bladder cancer patients using immunohistochemistry. *, P<0.05.

FIG. 10 illustrates results of confirming that migration and invasion of bladder cancer cells treated with conditioned media of STC1 overexpressing cells are promoted and secretory STC1 protein is expressed in bladder cancer cells. The left of FIG. 10A illustrates results of confirming STC1 protein in the conditioned media of control and STC1 overexpressing cells. The right of FIG. 10A illustrates an MTT assay result of bladder cancer cells treated with conditioned media of control and STC1 overexpressing cells. FIG. 10B illustrates improved colony formation ability of bladder cancer cells treated with conditioned media of STC1 overexpressing cells compared to control cells. FIG. 10C illustrates invasion and migration assay results of bladder cancer cells treated with conditioned media of control and STC1 overexpressing cells. FIG. 10D illustrates images of wound area (%) recovered after 24 hours of treatment with conditioned media derived from control and STC1 overexpressing cells on the wounded bladder cancer cells. The right of FIG. 10D illustrates results of Western blot of EMT markers in bladder cancer cells treated with conditioned media of control and STC1 overexpressing cells. FIG. 10E illustrates ELISA results of secretory STC1 protein expression in P0 and P15 cells, control and STC1 overexpressing vector-transfected cells, and control and STC1 overexpressing cells. FIG. 10F illustrates results of confirming secretory STC1 protein in the mouse serum identified in FIG. 10 through ELISA. **, P<0.01: ***, P<0.001.

FIG. 11 illustrates that secretory STC1 protein increases cell proliferation and metastatic ability in bladder cancer cells. FIG. 11A illustrates a result of confirming the proliferation of cells in P0 and P15 treated with recombinant human STC1 (rhSTC1) in media through MTT assay. FIG. 11B illustrates a result of confirming colony formation, cell invasion, and migration ability in P0 cells by adding rhSTC1 to the media. FIG. 11C illustrates that the invasion and migration ability of bladder cancer cells increased by rhSTC1 was neutralized by an STC1 antibody (ab). A right diagram illustrates a Western blot result of confirming p-FAK activation by rhSTC1. *, P<0.05: **, P<0.01.

FIG. 12 illustrates results of confirming a possibility as a biomarker by confirming secretory STC1 proteins in serum and urine samples of bladder cancer patients. FIG. 12A illustrates STC1 concentration confirmed through ELISA in serum samples from healthy subjects and bladder cancer patients. Scatter plots containing data are indicated to illustrate the range and distribution of measured levels of STC1 in serum by ELISA. The results of comparing bladder cancer patients divided by grade, stage, and recurrence are illustrated. FIG. 12B illustrates STC1 concentration confirmed through ELISA in urine samples from healthy subjects and bladder cancer patients. Scatter plots containing data are indicated to illustrate the range and distribution of measured levels of STC1 in urine by ELISA. The results of comparing bladder cancer patients divided by grade, stage, and recurrence are illustrated. ***, P<0.001.

### BEST MODE OF THE INVENTION

Hereinafter, examples of the present invention will be described in detail with reference to the accompanying drawings. In the following description, detailed descriptions of techniques well-known to those skilled in the art may be omitted. Further, in describing the present invention, the detailed description of associated known functions or constitutions will be omitted if it is determined that they unnecessarily make the gist of the present invention unclear. Further, terminologies used in the present specification are terminologies used to properly express examples of the present invention, which may vary according to a user, an operator's intention, or customs in the art to which the present invention pertains.

Accordingly, definitions of the terminologies need to be described based on contents throughout this specification. Throughout the specification, unless explicitly described to the contrary, when a certain part “comprises” a certain component, it will be understood to imply the inclusion of stated elements but not the exclusion of any other elements.

The present invention provides a novel Stanniocalcin-1 (STC1) biomarker for diagnosing or predicting the metastasis or prognosis of cancer.

“Stanniocalcin-1 (STC1)” of the present invention is a glycoprotein that is a homolog of stanniocalcin, a hormone first found in bony fish, and is encoded by a STC1 gene in humans and encodes a secreted homodimeric glycoprotein that is expressed in a wide variety of tissues and may have an autocrine or paracrine function. To date, as a known function of human STC1, only SUMO E3 ubiquitin ligase activity in a SUMOylation pathway has been reported. STC1 is known to interact with many proteins in the cytoplasm, mitochondria, endoplasmic reticulum, and cell nucleus.

As used in the present invention, the term “diagnosis” includes determining the susceptibility of a subject to a specific disease or disorder, determining whether a subject currently has a specific disease or disorder, determining the prognosis of a subject suffering from a specific disease or disorder (e.g., identifying a pre-metastatic or metastatic cancer condition, determining a stage of cancer, or determining the responsiveness of cancer to be treated), or therametrics (e.g., monitoring the condition of a subject to provide information about therapeutic efficacy).

The term “prognosis” refers to predicting various conditions of a patient due to cancer, such as the possibility of curing cancer, the possibility of recurrence after treatment, and the possibility of survival of a patient, after the cancer is diagnosed. The prognosis of cancer may be estimated from various aspects, but may be typically judged from aspects of recurrence probability, survival probability, and disease-free survival probability. For the purposes of the present invention, the prognosis may mean the prognosis of survival after diagnosis of cancer. Using the biomarker provided by the present invention, the survival prognosis of cancer patients can be more easily predicted, and can be used to classify patients into a high-risk group or to decide whether to use additional necessary treatment. This can contribute to increasing survival rates after developing cancer.

As used in the present invention, the term “(bio)marker, marker for diagnosis, or diagnosis marker” refers to a substance that may be determined to distinguish cancerous cells or tissues from normal cells or tissues, and includes organic biomolecules such as polypeptides or nucleic acids (e.g., mRNA, etc.), lipids, glycolipids, glycoproteins, and sugars (monosaccharides, disaccharides, oligosaccharides, etc.) that show an increased pattern in cancerous cells compared to normal cells.

Further, the present invention provides a biomarker composition for diagnosing or predicting the metastasis or prognosis of cancer, including an agent capable of measuring the expression level of Stanniocalcin-1 (STC1).

The composition measures the gene or protein expression level of STC1, and an agent used in a method for confirming the expression level of the gene or a fragment thereof refers to an agent used in a method for confirming the expression of the corresponding miRNA or a fragment thereof included in the sample. For example, the agent may be a primer, a probe or an antibody capable of specifically binding to a target gene used for methods such as RT-PCR, competitive RT-PCR, real-time RT-PCR, RNase protection assay (RPA), Northern blotting, gene chip analysis, etc., but is not particularly limited thereto.

As used in the present invention, the term “primer” refers to a nucleotide sequence having a short free 3′ hydroxyl group, and a short nucleotide sequence capable of forming base pairs with a complementary template and serving as a starting point for copying a template strand. The primer may initiate DNA synthesis in the presence of a reagent for polymerization (i.e., DNA polymerase or reverse transcriptase) and four different nucleoside triphosphates in appropriate buffer and temperature.

As used in the present invention, the term “probe” refers to a nucleic acid fragment, such as RNA or DNA, corresponding to several bases to several hundred bases capable of specifically binding to a gene or mRNA, and may be prepared in the form of an oligonucleotide probe, a single stranded DNA probe, a double stranded DNA probe, an RNA probe, etc., and may be labeled to be more easily detected.

The agent capable of measuring the expression level of the protein may include antibodies, aptamers, oligopeptides, or peptide nucleic acid (PNA) that specifically bind to the STC1, or primers, probes, or the like having a complementary sequence specific to the gene encoding the protein, but is not limited thereto.

According to an embodiment of the present invention, the STC1 may include a base sequence represented by SEQ ID NO: 1.

As used in the present specification, “polynucleotide” (or nucleotide, nucleic acid) has a meaning comprehensively including DNA (gDNA and cDNA) and RNA molecules, and nucleotides, which are basic structural units in nucleic acid molecules, include not only natural nucleotides but also analogues with modified sugar or base sites.

The polynucleotide of the present invention is not limited to nucleic acid molecules encoding a specific amino acid sequence (polypeptide), and is interpreted to include a nucleic acid molecule encoding an amino acid sequence showing substantial identity to a specific amino acid sequence or a polypeptide having a corresponding function thereto.

According to an embodiment of the present invention, the cancer may be selected from the group consisting of bladder cancer, breast cancer, glioblastoma, prostate cancer, cerebrospinal tumor, head and neck cancer, lung cancer, thymoma, mesothelioma, esophageal cancer, gastric cancer, colon cancer, liver cancer, pancreas cancer, biliary tract cancer, kidney cancer, testicular cancer, germ cell tumor, ovarian cancer, cervical cancer, endometrial cancer, lymphoma, acute leukemia, chronic leukemia, multiple myeloma, sarcoma, malignant melanoma and skin cancer.

According to an embodiment of the present invention, when the expression of STC1 is increased compared to a reference value of the control group, the growth, invasion, or migration of cancer cells may be increased.

According to an embodiment of the present invention, when the expression of STC1 is increased compared to the reference value of the control group, the clinical stage of cancer may be increased.

A method for estimating diagnosis and prognosis of cancer according to the present invention may be used to determine the severity (clinical stage) of cancer. For example, compared to the profiles of positive and negative controls, the severity (clinical stage) of cancer may be assessed as mild, moderate or severe. Furthermore, marker profile analysis may be performed on a certain cancer group and the cancer group may be classified according to certain criteria based on the profile results.

According to an embodiment of the present invention, the STC1 may be measured in a sample isolated from a subject, and the sample may be selected from the group consisting of tissue, cells, whole blood, serum, plasma, saliva, sputum, cerebrospinal fluid, and urine, preferably serum or urine, but is not limited thereto.

Further, the present invention provides a kit for diagnosing or predicting the metastasis or prognosis of cancer including the composition.

The term “kit” as used herein refers to a set of a composition and components required for a specific purpose. For the purpose of the present invention, the kit of the present invention is to confirm the diagnosis or prognosis of cancer. The kit of the present invention may include primers and probes for confirming the diagnosis or prognosis of cancer, antibodies that selectively recognize peptides or antibodies that recognize specific peptides with expression specifically changed during cancer development, and one or more different component compositions, solutions or devices suitable for an analysis method.

Further, the present invention provides an information providing method for diagnosing and predicting the metastasis or prognosis of cancer including: isolating a biological sample from a subject;
measuring the expression level of Stanniocalcin-1 (STC1) in the isolated biological sample; and
comparing the expression level of STC1 with a reference value of a control group.

According to an embodiment of the present invention, the biological sample may be selected from the group consisting of tissue, cells, whole blood, serum, plasma, saliva, sputum, cerebrospinal fluid, and urine.

According to an embodiment of the present invention, when the expression of STC1 is increased compared to the reference value of the control group, it may be judged to be cancer.

According to an embodiment of the present invention, when the expression of STC1 is increased compared to the reference value of the control group, it is determined that the growth, invasion, or migration of cancer cells may be increased.

According to an embodiment of the present invention, when the expression of STC1 is increased compared to the reference value of the control group, it is determined that the clinical stage of cancer may be increased.

Further, the present invention provides a screening method of an anticancer agent including: isolating a biological sample from a subject;
treating the isolated biological sample with a candidate substance;
measuring the expression level of Stanniocalcin-1 (STC1) in the biological sample treated with the candidate substance; and
comparing the expression level of STC1 with a reference value of a control group.

According to an embodiment of the present invention, when the expression of STC1 is low compared to the reference value of the control group, it may be determined to have an anticancer effect.

Further, the present invention provides a method for diagnosing and predicting the metastasis or prognosis of cancer including: isolating a biological sample from a subject;
measuring the expression level of Stanniocalcin-1 (STC1) in the isolated biological sample; and
comparing the expression level of STC1 with a reference value of a control group.

### MODES FOR THE INVENTION

Hereinafter, the present invention will be described in more detail through Examples. These Examples are to explain the present invention in more detail, and it will be apparent to those skilled in the art that the scope of the present invention is not limited to these Examples.

### Example 1. Preparation for Discovery and Functional Evaluation of Novel Biomarkers

### 1. Cell Culture

Human bladder cancer cell lines T24, 5637, UC3, UC5, and UC14 were purchased from American Type Culture Collection (ATCC), and an RT4 cell line was purchased from Korean Cell Line Bank (KCLB). The T24, UC3, UC5, UC14, and RT4 cell lines were cultured in a DMEM (Dulbecco's modified Eagle's medium), and the 5637 cell line was cultured in RPMI 1640 added with 10% FBS (Capricorn Scientific GmbH, Ebsdorfergrund, Germany) and 1% penicillin/streptomycin (Capricorn Scientific GmbH, Ebsdorfergrund, Germany). All of the cell lines were cultured at 37° C. in a humidified atmosphere of 5% CO2.

2. Harvest of Proteins Secreted from Conditioned Medium (CM)

A serum-free conditioned medium (CM) was prepared as T24 (P0) and P15 (150 mm dishes) cultured in 40 ml of a serum-free medium for 6 hours. The medium was collected and cell debris was removed at 1,000 rpm for 10 minutes. The conditioned medium was concentrated with VIVASPIN (GE Healthcare, USA) at 3,850 rpm for 2 hours at 4° C.

### 3. Proteomic Analysis by LC-MS/MS

A protein concentration was confirmed by BCA assay and samples were stored at −70° C. for further studies. 10 μg of a protein sample was separated on 12% SDS-PAGE gel, and this gel was stained with a Coomassie Brilliant Blue R-250 buffer. In-gel digestion was constructed according to a method described in previous literature [Schevchenko A. et al., Nature Protocols 2006; 1(6)2856-2860]. The gel was divided into four parts according to a molecular weight. After desalting the gel fraction, the cysteine of the protein was reduced and alkylated, and then degraded to trypsin. The degraded peptide was extracted with an extraction solution buffer. The degraded peptide was dissolved in 10 μl of a sample solution containing 0.02% formic acid and 0.5% acetic acid. LC-MS/MS analysis was performed at least three times for each sample.

4. Construction of STC1 Overexpressing Vector and Knockdown Using Small-Interference RNAs (siRNA)

For transfection of a plasmid expression vector encoding human STC1, the cDNA sequence encoding STC1 was cloned by RT-PCR from normal human tissue as a substrate, and the PCR product was subcloned with a pcDNA/His B vector. DNA sequence containing a STC1 open reading frame at the side of a HindIII-BamHI restriction site was PCR-amplified from T24 cells. For knockdown of endogenous STC1, the cells were transfected with siSTC1 oligonucleotide. The siSTC1 oligonucleotide was purchased from Dharmacon SMARTPool. Scrambled siRNA (scRNA) or siSTC1 transfection was performed at a final siRNA concentration of up to 100 nM. Knockdown efficiency was confirmed using qRT-PCR or Western blot analysis, respectively.

### 5. MTT and Colony Formation Assay

1×10³ cells were cultured in each well of a 96-well plate. 3-(4,5-dimethylthiazol-2-yl)-2,5-diphenyltetrazolium bromide (MTT) was added to each well, incubated for 1 hour, and added with dimethyl sulfoxide (DMSO). Absorbance at 540 nm was measured using a spectrophotometer microplate reader, and cell viability was calculated as a percentage compared to control cells.

1×10³ cells were cultured in each well of a 6-well plate and cultured for up to 7 days until visible colonies were formed. The colonies were fixed with 4% formaldehyde for 10 minutes and stained with a 0.1% crystal violet solution for 1 hour. The colony numbers were counted manually using Image J software.

### 6. Cell Invasion and Migration Assay

The invasion ability of cells was measured in a Boyden chamber using a Transwell assay. 4×10⁴ cells were loaded into a matrigel-coated chamber and then cultured for 24 hours. In the case of cell invasion analysis by a conditioned medium (CM) of cells, in order to confirm the invasion or migration ability of cells by CM, the cells were treated in basic composition and conditioned medium at a 1:1 ratio for 24 hours to confirm invasion or migration ability.

### 7. Wound Healing Assay

Cells were inoculated in a 6-well plate and cultured for 24 hours until 90% confluent. After creating a wound on the surface of the plate with a yellow tip of a P200 pipette, the cells were washed several times with PBS to remove cell debris, and the cells were cultured at 37° C. in 5% CO2. After 24 hours, the cells were visualized by light microscopy. Thereafter, photographs of the wounded area were taken at intervals. Three random fields were marked and measured. The migration index was expressed as a ratio of the migration distance of treated cells to that of control cells.

8. Quantitative Real-Time Polymerase Chain Reaction (qRT-PCR)

Total RNA was isolated using an RNAiso reagent (Takara). RNA quantitative check was evaluated using a spectrophotometer (ND-1000). Primary strand cDNA synthesis was performed from 1 μg of total RNA using a PrimeScript™ RT reagent Kit (Takara). qRT-PCR was performed using TB Green Premix Ex Taq (Takara) and CFX 96 real-time PCR Detection system (BioRad). The primer set sequences used were shown in Table 1. The reproducibility of the quantitative evaluation was evaluated by three independent cDNA syntheses and PCR amplification from each preparation of RNA. For mRNA analysis, data were normalized to GAPDH as an endogenous control and fold change was calculated via relative quantification (2^{-ΔΔCt}).

TABLE 1

| | Forward Primer | Reverse Primer |
|---|---|---|
| Gene | Sequences | Sequences |
| GAPDH | TGCACCACCAACTGCTTAGC | GGCATGGACTGTGGTCATGAG |
| MMP1 | TTTGGCTTCCCTAGAACTGT | GCTATCATTTTGGGATAACCT |
| | G | GG |
| MMP2 | GCGGCGGTCACAGCTACTT | CACGCTCTTCAGACTTTGGTT |
| | | CT |
| MMP9 | CCTGGAGACCTGAGAACCAA | CCACCCGAGTGTAACCATAGC |
| | TC | |
| VIM | AGGCAAAGCAGGAGTCCACT | ATCTGGCGTTCCAGGGACTCA |
| | GA | T |
| SNAIL | CCACAAGCACCAAGAGTC | TGGCAGTGAGAAGGATGT |
| SLUG | TTCACTCCGAAGCCAAATG | TCTCTCTGTGGGTGTGTG |
| ZEB1 | TGTGCCAATTTGTTCCTGTA | TGAGATGGGAGTCTGGTAAA |
| ZEB2 | ATCGTGTAACAAAGATGAAG | TCACAAATGTCTCAAGTTCTA |
| | AAA | AA |
| TWIST | GCCAGGTACATCGACTTCCT | TCCATCCTCCAGACCGAGAAG |
| | CT | G |
| NCAD | GAATTCAGCACCCCCCTCAG | GCTGCATATATCGATCTGGG |
| SDC1 | TTCACACTCCCCACACAGAG | ACTACAGCCGTATTCTCCCC |
| SDC2 | TGTACCTTGACAACAGCTCC | CTCTACATCCTCATCAGCTCC |
| ECAD | GCAGTGACGAATGTGGTACC | GTGTCTGGCTCCTGGGCAGT |
| STC1 | AGCGCTGCTAAATTTGACAC | CTTTGGAAAGTGGAGCACCTC |
| | T | CG |

### 9. Western Blot Analysis and Antibodies

Western blot analysis was performed according to the manufacturer's instructions. Cells was first washed with PBS, and then the proteins were isolated with a radio immunoprecipitation (RIPA) buffer (Ambion, 150 mM NaCl, 0.5% sodium deoxycholate, 0.1% SDS, 50 mM Tris, pH 8.0, protease inhibitor cocktail, and phosphatase inhibitor) and centrifuged (12,000 g, 15 min, 4° C.). The amount of proteins was evaluated using a BCA assay kit (Thermo Fisher Scientific), subjected to 10-12% SDS-polyacrylamide gel electrophoresis (SDS-PAGE), and then electrically transferred to a nitrocellulose (NC) membrane (GE healthcare). The membrane was then blocked using 5% fat-free milk in 0.05% TBS-T. Primary antibodies for each target were used as follows: STC1 (Santa Cruz Biotech), MMP-1 (Santa Cruz Biotech), MMP2 (Cell signaling), MMP9 (Cell signaling), NCAD (Cell signaling), ECAD (Cell signaling), VIM (Santa Cruz Biotech), SNAIL (Santa Cruz Biotech), FAK (Cell signaling), p-FAK (Cell signaling), ERK (Cell signaling), and p-ERK (Cell signaling). GAPDH (Cell signaling) was used as a loading control. Horseradish peroxidase (HRP)-conjugated anti-rabbit or anti-mouse immunoglobulin G (IgG) was used as a secondary antibody, and positive bands were detected using an ECL detection reagent. The final visualization of a chemifluorescence signal was captured with an automatic X-ray film processor (JPI Healthcare), and the signal intensity on X-ray films (Fuji film) was quantified with Image J software.

### 10. In Vivo Tumor Growth and Metastasis Ability Assay

For in vivo tumor formation and metastasis ability assay, P0 or P15 cells were trypsinized and suspended in PBS. Thereafter, cells were injected subcutaneously into the lateral and intravenously into the tail veins of each BALB/C nude mouse. To confirm tumor formation ability, the cells were mixed with 200 μl of cells in PBS and an equal amount of Matrigel and injected subcutaneously. When the mouse's body weight and tumor were measured using calipers at a measurable time and the tumor volume was calculated: Tumor volume (mm³)=width² (mm²)×length (mm). When extracting RNA and protein from tumor tissue, the mouse tissue was washed twice with PBS, and on day 35 of measurement, the mouse was dissected to obtain the tissue. To confirm tumor metastasis ability, the mouse injected into the tail vein was dissected to determine the number of lung nodules formed.

### 11. Tissue Microarray (TMA) and Immunohistochemistry (IHC)

TMA blocks were selected from paraffin-blocks of the mouse with a tissue diameter of 2 mm. Slides were stained with hematoxylin and eosin (H&E) and observed to identify representative tumor tissues. For IHC, all tissue samples were fixed in buffered formalin (Sigma-Aldrich, St. Louis, MO, USA) and impregnated in paraffin. Paraffin-impregnated tissues were deparaffinized in xylene and rehydrated in alcohol (100%, 90%, 80%, and 60%). Antigen recovery (10 minutes in boiling water) was performed and sodium citrate was used as a pH 7 recovery buffer. The primary STC1 and Ki67 antibodies (Santa Cruz Biotech) were used. A TMA slide was treated at 4° C. with a primary antibody and treated with a biotinylated secondary antibody. The slide was added with a Vectastain Elite ABC Reagent (Vector Laboratories) at room temperature for 30 minutes, and the immune response was detected using 3,3′-diaminobenzidine (DAB) as a chromogen. Thereafter, the TMA slide was counterstained with Mayer's hematoxylin (Dako), dehydrated with alcohol (60%, 80%, 90%, and 100%), washed three times with xylene, and fixed with an encapsulant in xylene. The staining results were confirmed under a microscope.

### 12. Human Serum and Urine Samples

The blood from BC patients was collected in a heparin-added saline tube and centrifuged at 3,000 rpm for 10 minutes. Serum isolated from the blood was frozen and stored. Urine samples were collected from healthy subjects and bladder cancer patients, respectively. 20 ml of urine in the tube was centrifuged at 3,000 rpm for 10 minutes at 4° C. The supernatant of urine was concentrated using a VIVASPIN column and used in the experiment.

### 13. Human STC1 ELISA Assay

The concentrations of STC1 in conditioned media, serum, and urine samples were analyzed using an enzyme-linked immunosorbent assay (ELISA) kit (R&D systems).

### 14. Patient and Gene Expression Data

Data sets including clinical and gene expression data were obtained from the National Center for Biotechnology information (NCBI) Gene Expression Omnibus (GEO) database (GSE13507, GSE32894, and GSE120736). All data were transformed to log 2 scale and normalized by quantile normalization. Data for 165 bladder cancer patients were used as a discovery cohort (n=165; Korean cohort; GSE13507), and data for 453 bladder cancer patients were used as a validation cohort (n=308: Lund cohort: GSE32894, n=145; Yonsei cohort: GSE120736).

### 15. Association, Gene Expression and Function Enrichment Analysis

To prepare a significant gene set associated with genetic characteristics, a Pearson correlation test was applied to gene expression data from the Korean bladder cancer patient cohort (GSE13507) and genes with significant correlation coefficients (|r|>0.4 and p<0.001) were selected. Hierarchical clustering analysis was performed with central correlation coefficients as a measure of a similarity and complete linkage clustering method. According to the patient clustering results, patients were divided into two subgroups, and the progression time and cancer specific survival rate of patients in each subgroup were evaluated. Progression-free survival and cancer specific survival were calculated with log-rank statistics using a Kaplan-Meier method. Gene ontology (GO) analysis was performed with DAVID bioinformatic resources (http://david.ncifcrf.gov), and results were considered significant when p<0.001 and false discovery rate (FDR)<0.25.

### 16. Statistical Analysis

Data results were shown as mean±standard deviation (SD) of three repeat studies. All analyses were performed at least three times and were presented as data from three separate experiments. All numerical data were expressed as mean±S.D. The significance in difference between two independent groups was determined using a two-tailed Student's t-test. The difference was considered statistically significant at P<0.05. *, P<0.05: **, P<0.01: ***, P<0.001. Statistical analysis was performed using an R 3.6.1 language environment (http://www.r-project.org).

### Example 2. Discovery of Novel Biomarkers and Evaluation of Functions Thereof

1. Analysis of Proteins Secreted from Conditioned Media (CM) of Anticancer Drug-Resistant Bladder Cancer Cells

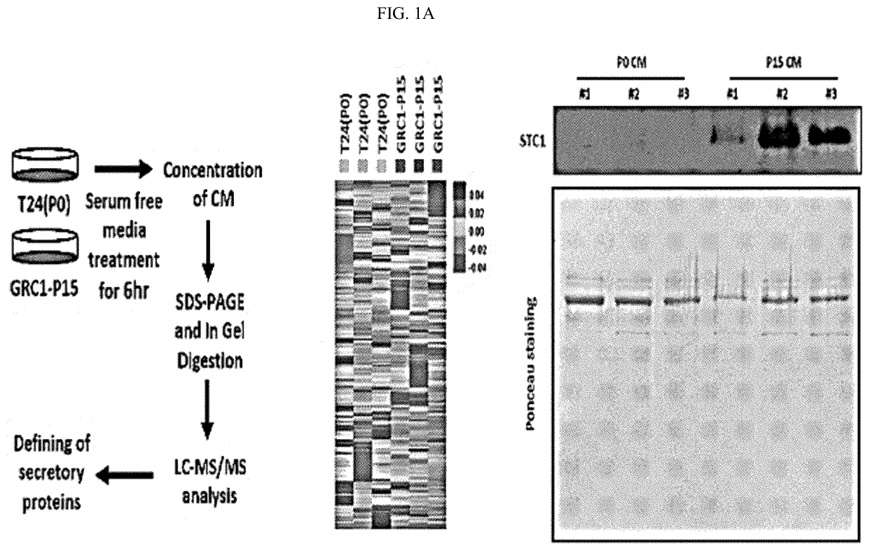

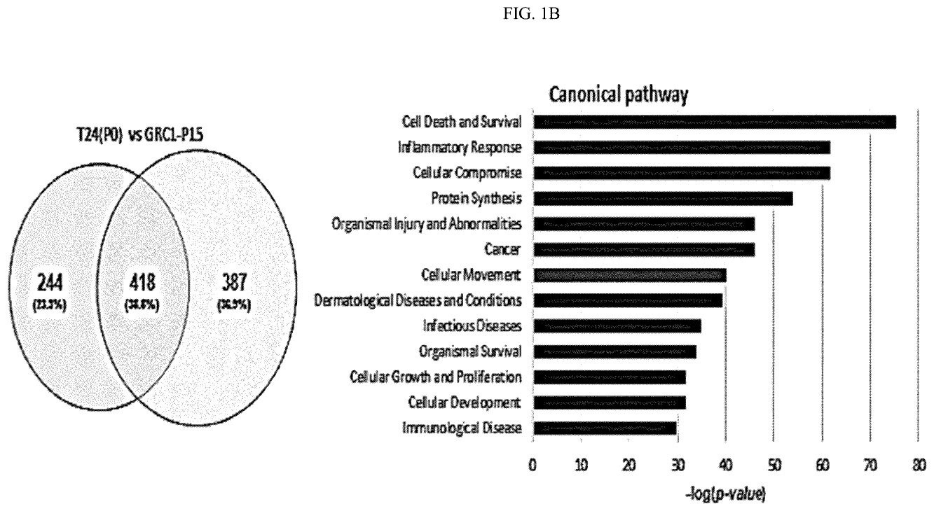

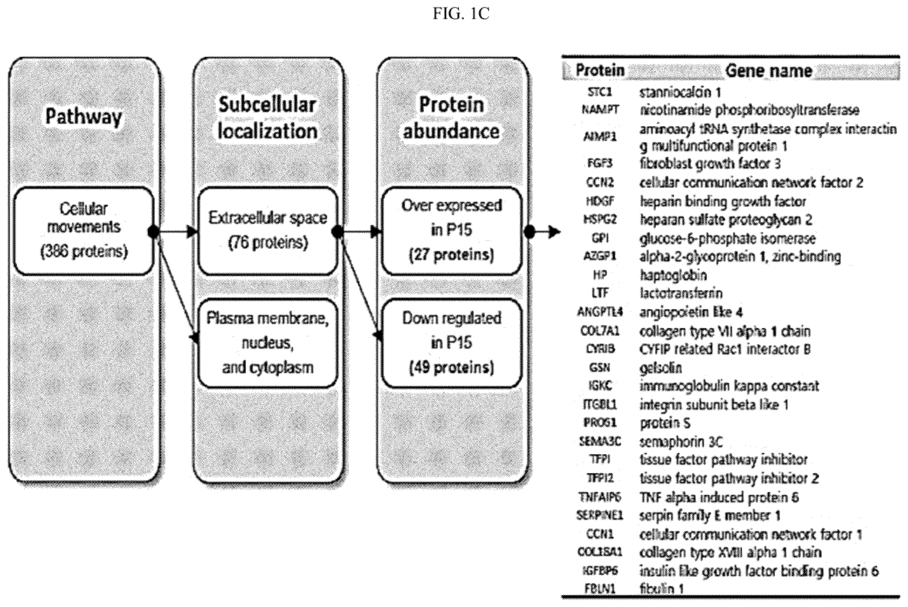

To identify proteins secreted from anticancer drug-resistant bladder cancer cell lines, samples were prepared from conditioned media of P0 and P15 cells and then liquid chromatograph-tandem mass spectrophotometer (LC-MS/MS) was performed (FIG. 1A). A large number of proteins expressed in the conditioned media of P0 and P15 cells were identified, and 662 and 805 secretory proteins were identified, respectively (FIG. 1B). The proteins secreted only from the conditioned media of P15 cells compared to P0 were analyzed, and related pathways were identified. As a result, the proteins were analyzed by ingenuity pathway analysis (IPA), and it was found that the expression of proteins related to cell migration changed significantly in 13 reference pathways (FIG. 1B). Among the 386 proteins differentially expressed between the conditioned media of P0 and P15 cells, a total of 27 proteins that were overexpressed in P15 cells and located in an extracellular space were identified (FIG. 1C).

### 2. Usability of STC1 as Biomarker in Bladder Cancer Cells and Other Cancer Cells

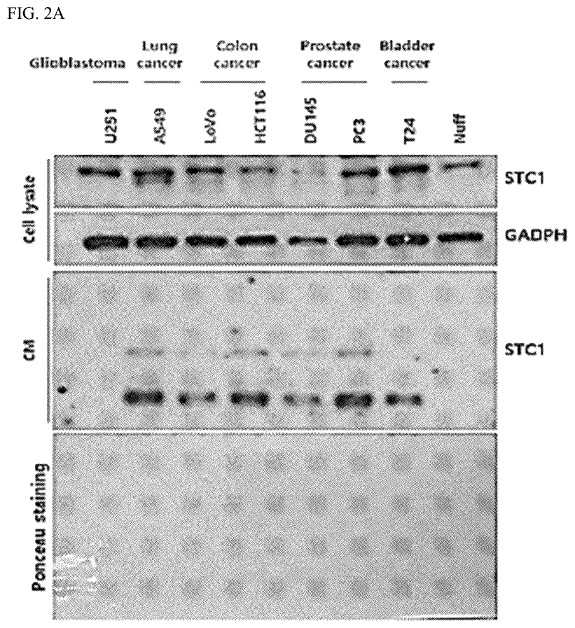

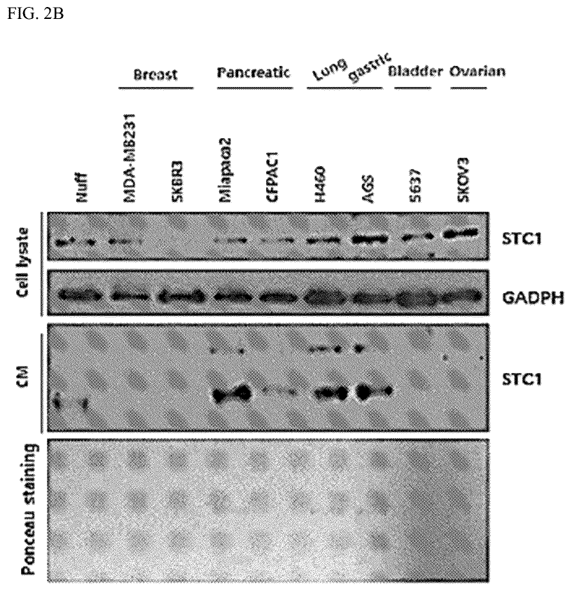

Next, the expression levels of STC1 were confirmed not only in bladder cancer but also in various cancer types. Specifically, in glioblastoma (U251), lung cancer (A549, H460), colon cancer (LoVo, HCT116), prostate cancer (DU145, PC3), bladder cancer (T24, 5637), breast cancer (MDA-MB231, SKBR3), pancreatic cancer (Miapaca2, CFPAC1), gastric cancer (AG5), and ovarian cancer (SKOV3) cell lines, STC1 proteins in cell lysates and CM were quantified by Western blotting. As a control group, human Newborn foreskin fibroblasts (Nuff) were used. As a result, STC1, which was expressed in bladder cancer, was also identified to be expressed in various cancer types (glioblastoma, lung cancer, colon cancer, prostate cancer, breast cancer, pancreas cancer, gastric cancer, and ovarian cancer) including bladder cancer, and was identified as a cancer cell-specific marker (FIGS. 2A and 2B).

### 3. Confirmation of Association Between High STC1 Expression and Poor Prognosis in Bladder Cancer Patients

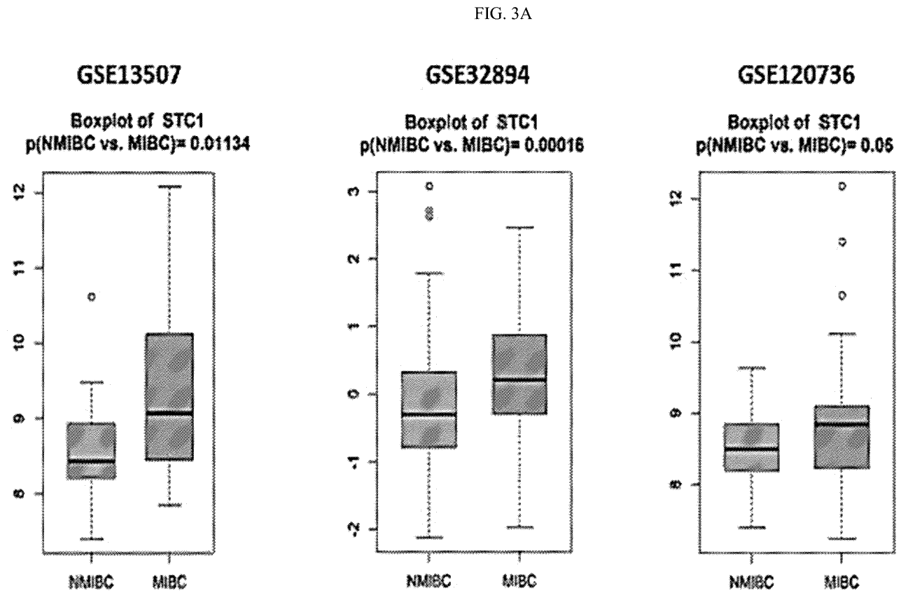

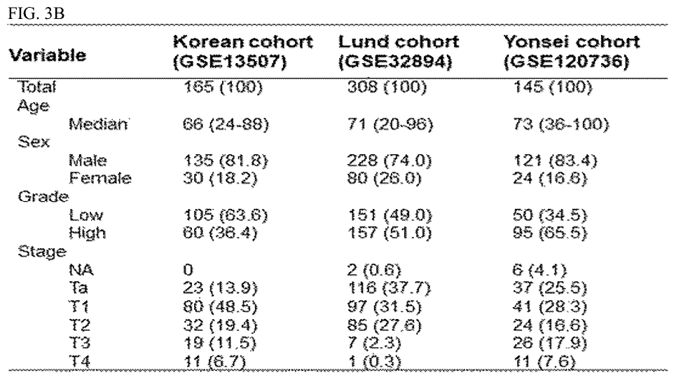

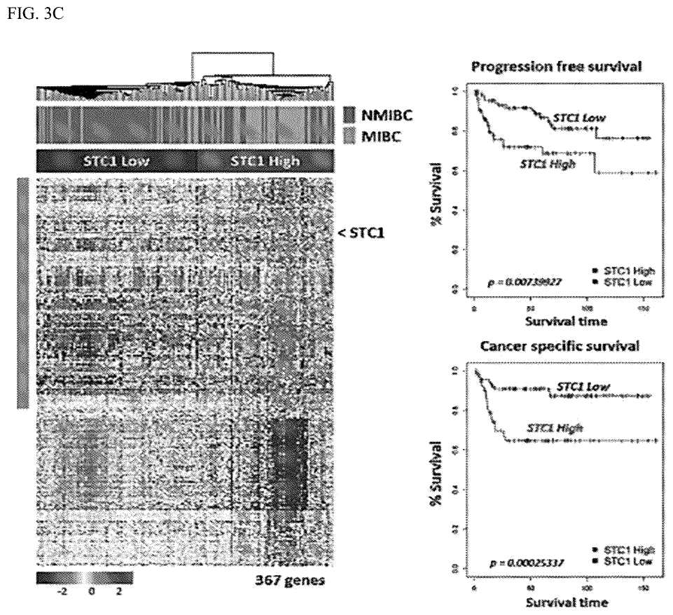

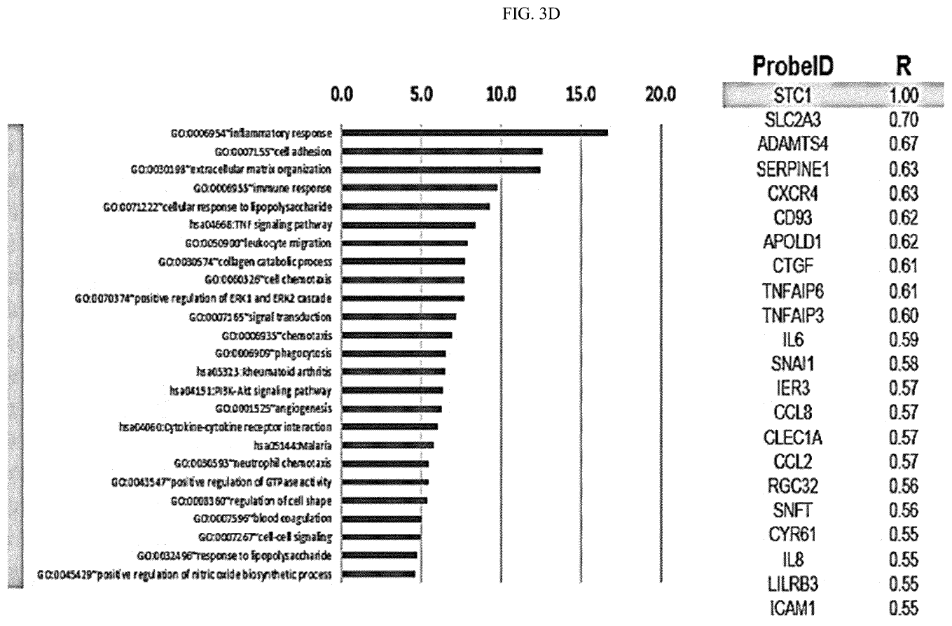

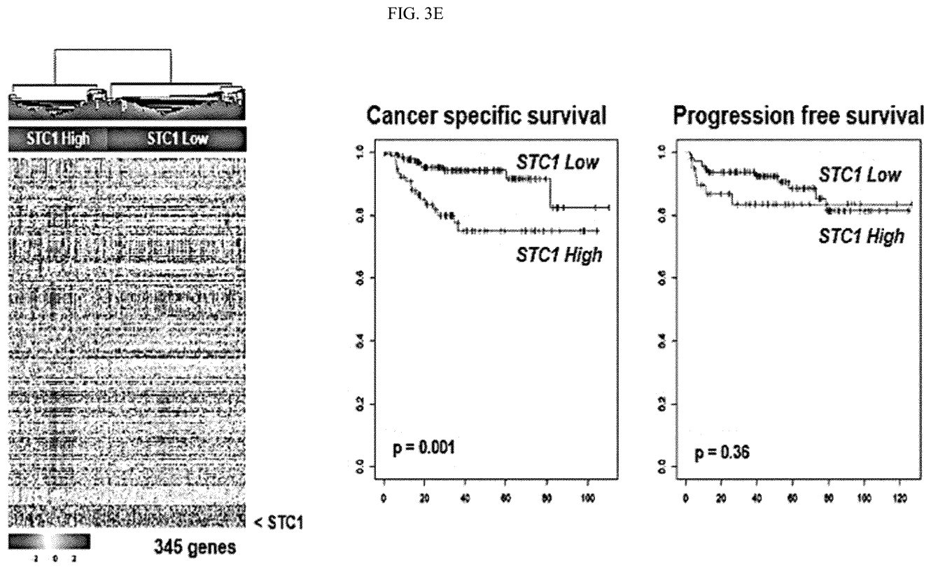

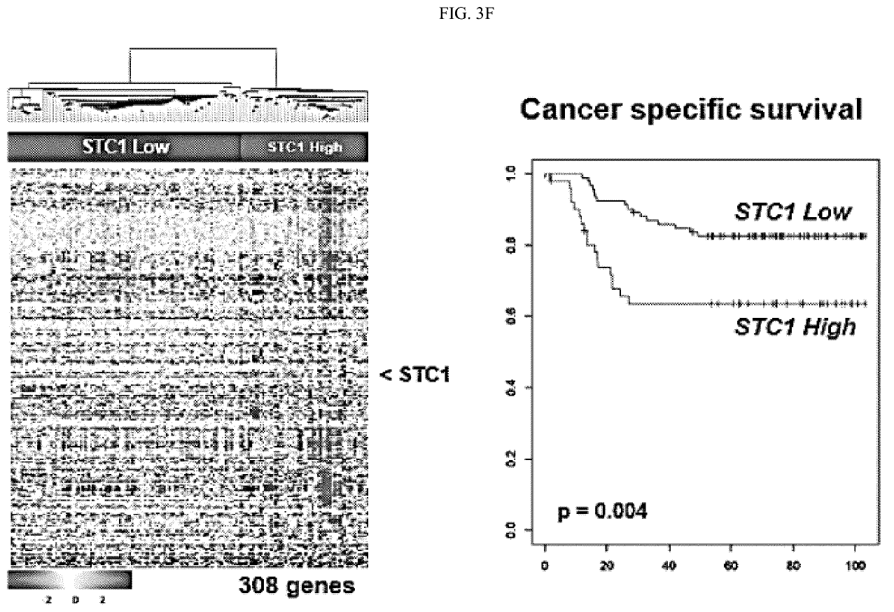

First, the gene expression level of STC1 was confirmed and compared with the expression level in bladder tissue including primary NMIBC, primary MIBC, and recurrent tissue in the bladder cancer cohort. In comparison of gene expression data in various bladder cancer cohorts (Korean bladder cancer cohort, GSE13507; Lund cohort, GSE32894; Yonsei cohort, GSE120736), in all cases, the expression level of STC1 in primary MIBC was significantly higher than that in primary NMIBC (P=0.01, P<0.001, and P=0.05 by a two-sample t-test, FIG. 3A). FIG. 3B illustrates basic characteristics of 618 bladder cancer patients. In the Korean bladder cancer cohort (GSE13507), the mean age was 66 years (range from 24 to 88 years) and the mean subsequent period after surgery was 53 months (range from 1 to 161 months). During the subsequent period, 34 patients (21%) had developed the disease. In the Lund cohort, the mean age was 71 years (range from 20 to 96 years) and the mean subsequent period after surgery was 46 months (range from 2 to 127 months). During the subsequent period, 19 patients (12%) had developed the disease. In the Yonsei cohort, the mean age was 73 years (range from 36 to 100 years) and the mean subsequent period after surgery was 70 months (range from 1 to 103 months). However, progression-free survival data were not supplied from the Yonsei cohort.

Since the STC1 was commonly upregulated in many cancers and used as a prognostic marker, the expected level of STC1 was further evaluated in the survival results of bladder cancer patients. This was to identify a gene expression signature directly related to the STC1 expression level and to be used as a signature for predicting disease progression and survival probability. In the GSE13507 cohort, 367 genes related to STC1 expression were identified (Pearson's correlation test, P<0.001, |r|>0.4). Based on hierarchical clustering analysis of the expression patterns of these genes, bladder cancer patients were divided into two groups of STC1-low and STC1-high (FIG. 3C). The progression-free survival rate of STC1-low patients was significantly higher than that of STC1-high patients (log-rank test, P=0.007; FIG. 3C). The cancer specific survival rate of STC1-low patients was significantly higher than that of STC1-high patients (log-rank test, P=0.001; FIG. 3C). In the present invention, important signaling pathways related to STC1 expression were also identified. GO analysis of 367 STC1-related genes (279 upregulated genes) was performed using DAVID software. When the upregulated genes were applied to DAVID, genes related to inflammatory response, extracellular matrix organization, leukocyte migration, cell chemotaxis, signal transduction, phagocytosis, and PI3K-Akt signaling pathways were significantly enriched (FIG. 3D). In addition, 21 genes closely related to STC1 expression were also identified (FIG. 3D). The findings were validated using gene expression data from independent patient cohorts (GSE32894 and GSE120736). Bladder cancer patients were classified into STC1-high or STC1-low groups through the same process as the signature-based hierarchical cluster analysis in the Lund cohort (GSE32984) (FIG. 3E). The progression-free survival rate of STC1-low patients was significantly higher than that of STC1-high patients (log-rank test, P=0.001 and P=0.36; FIG. 3E). In addition, bladder cancer patients were classified into two groups in the Yonsei cohort (GSE120736). The cancer specific survival rate of STC1-low patients was significantly higher than that of STC1-high patients (log-rank test, P=0.004; FIG. 3F).

### 4. Confirmation of Promotion of Cell Proliferation, Migration, and Invasion Ability by STC1 in Bladder Cancer

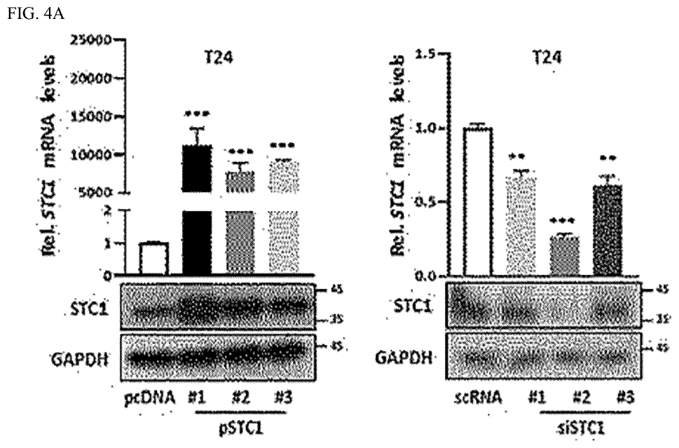

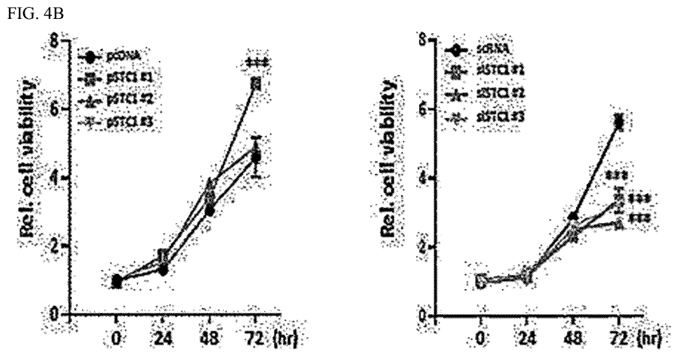

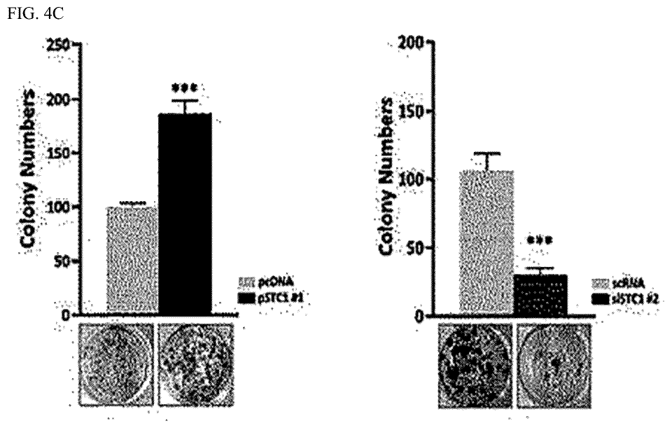

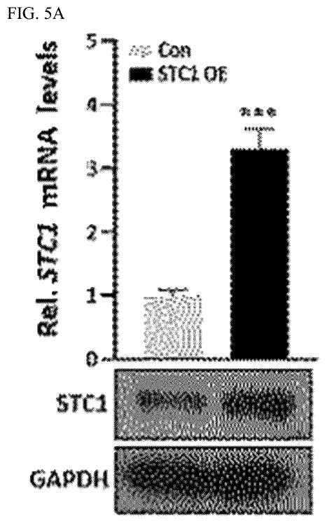

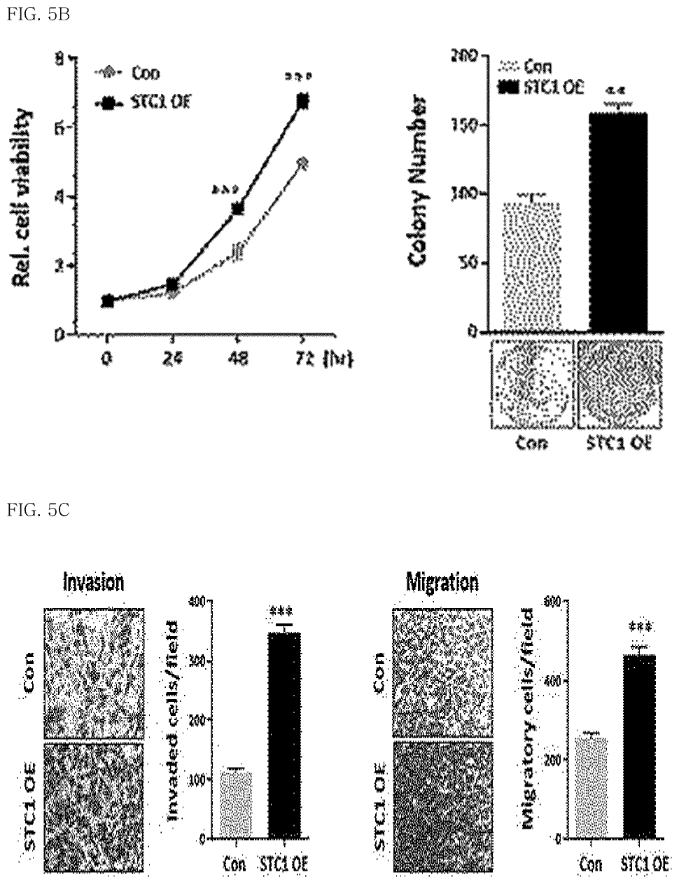

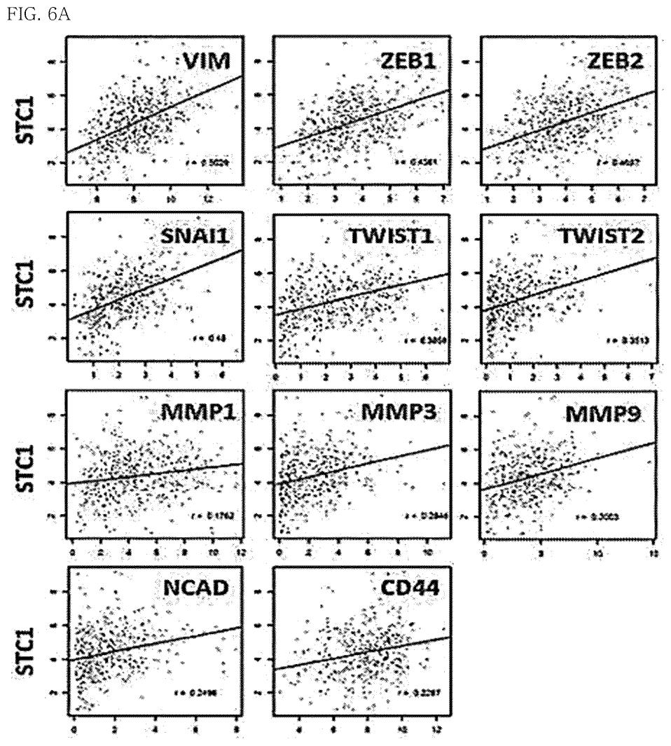

Increased STC1 expression was associated with poor prognosis in patients with various types of cancer. To determine whether STC1 contributed to cell proliferation in bladder cancer, an increase and decrease in expression was first confirmed in the P0 cell line by cell lines transfected with a STC1 overexpressing vector (pSTC1) or STC1 small-interference RNA (siSTC1) (FIG. 4A). Using cell lines overexpressing and knocking down STC1, cell proliferation through MTT assay (FIG. 4B) experiment and colony formation ability through clonogenic assay (FIG. 4C) were confirmed. In the two results, it was confirmed that cell proliferation was regulated by the expression of STC1 (FIGS. 4B and 4C). In addition, even in STC1 overexpressing stable (STC1 OE) cell lines, compared to control cells, it was confirmed that STC1 mRNA and protein expression, cell proliferation, colony formation ability, invasion and migration ability were all increased (FIGS. 5A, 5B, and 5C). Through these results, it was suggested that STC1 promoted not only cell proliferation but also migration and invasion ability in bladder cancer.

5. Confirmation of Correlation of STC1 with Epithelial-Mesenchymal Transition (EMT) Genes

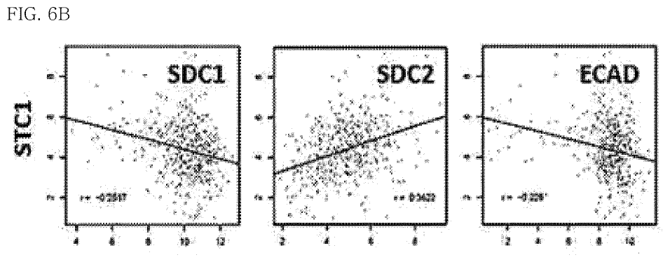

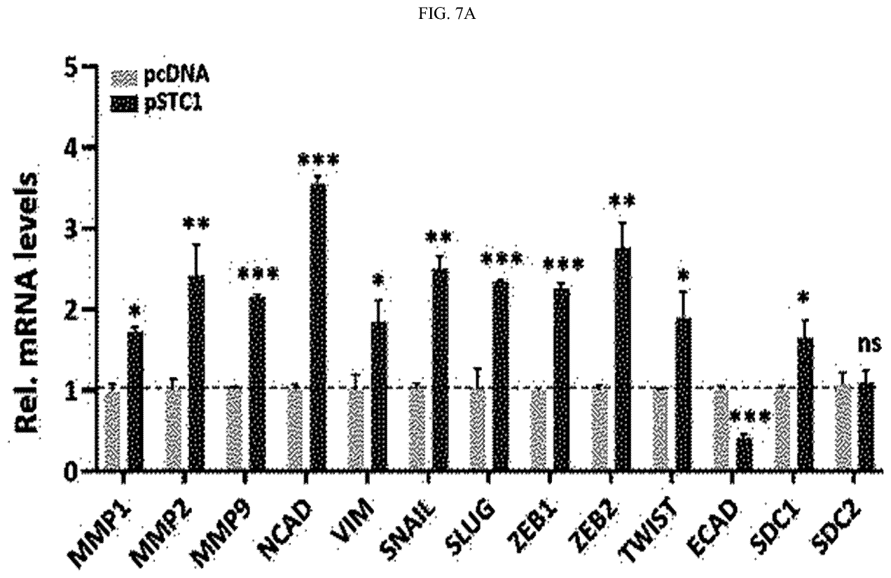

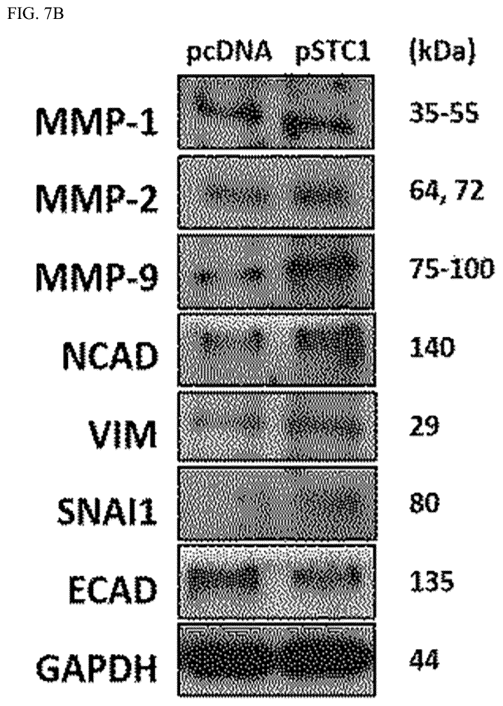

In GSE13507 data, EMT-related genes VIM, ZEB1, ZEB2, SNAI1, TWIST1, TWIST2, MMP1, MMP3, MMP9, NCAD, and CD44 showed a positive correlation with STC1 (FIG. 6A) and mesenchymal-epithelial transition (MET)-related genes SDC1 and ECAD showed a negative correlation with STC1 (FIG. 6B). Next, in order to determine whether STC1 regulated the expression of genes related to migration and EMT in bladder cancer cells, it was confirmed that the mRNA expression of MMP1, MMP2, MMP9, NCAD, VIM, SNAIL, SLUG, ZEB1, ZEB2, and TWIST was increased (FIG. 7A). Through Western blotting results, it was confirmed that the protein levels of MMP1, MMP2, MMP9, NCAD, VIM, and SNAIL were increased, and on the other hand, it was confirmed that the MET-related ECAD protein level was decreased (FIG. 7B).

### 6. Confirmation of Increased Bladder Cancer Tumor Growth and Lung Metastasis by STC1 In Vivo

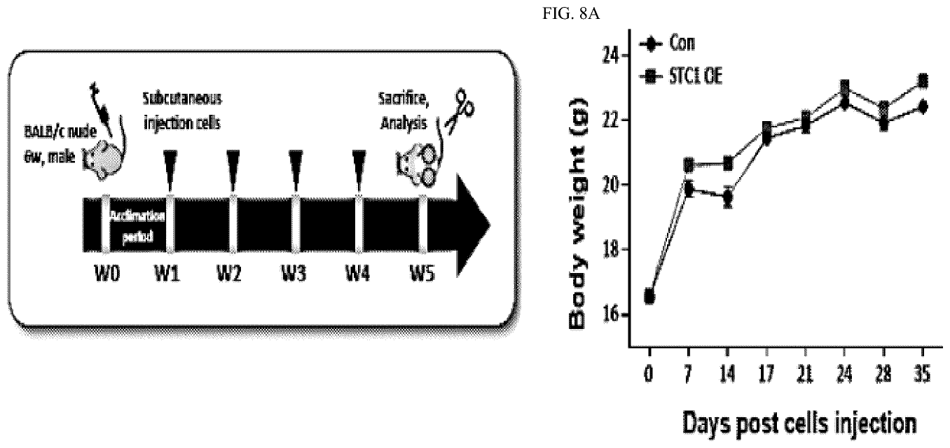

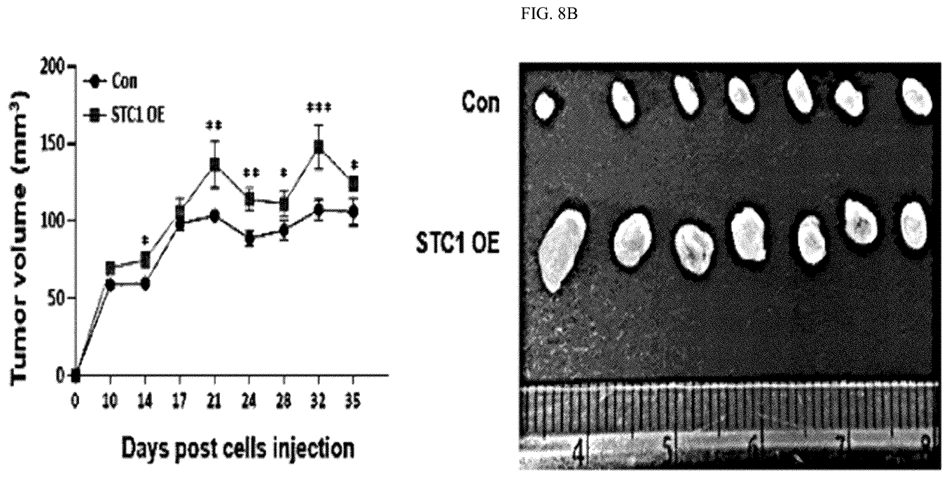

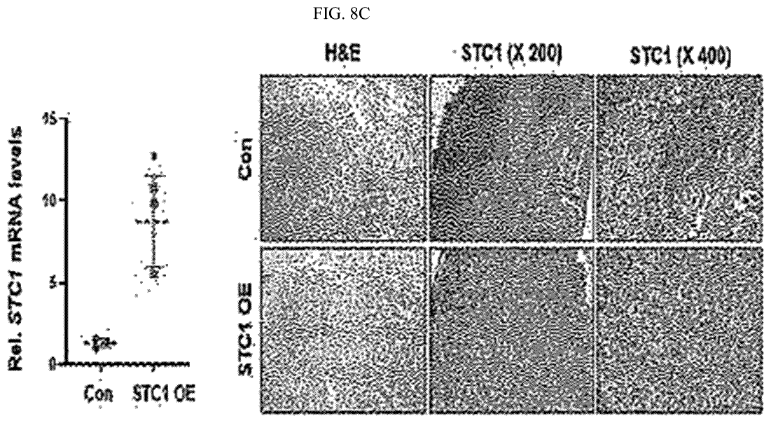

Control cells and STC1-overexpressing stable cells were injected subcutaneously into the flank area of male BALB/C nude mice, and an overall experimental schematic diagram was as follows (FIG. 8A). The body weight of a mouse group injected with STC1-overexpressing stable cells increased, although not significantly, compared to a control mouse group, and the tumor size increased rapidly (FIGS. 8A and 8B). FIG. 8B shows representative photographs of tumors formed on the flanks of mice injected with control and STC1-overexpressing stable cells. The mRNA expression of STC1 was more increased in the STC1 overexpressing stable mouse group than in the tumors of the control cell-injected mouse group (FIG. 8C). Therefore, the protein expression of STC1 identified by IHC was increased in mouse tumors injected with the STC1 overexpressing stable cell line (FIG. 8C). These results showed that STC1 increased tumor growth in vivo.

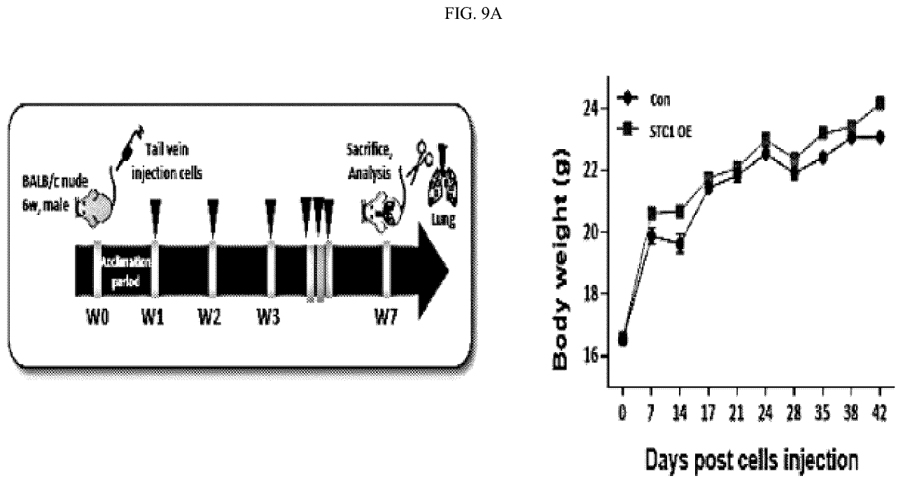

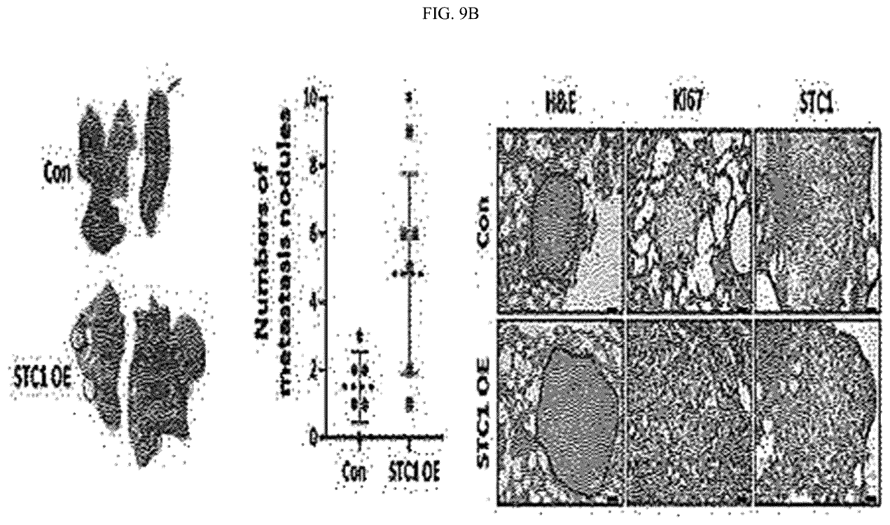

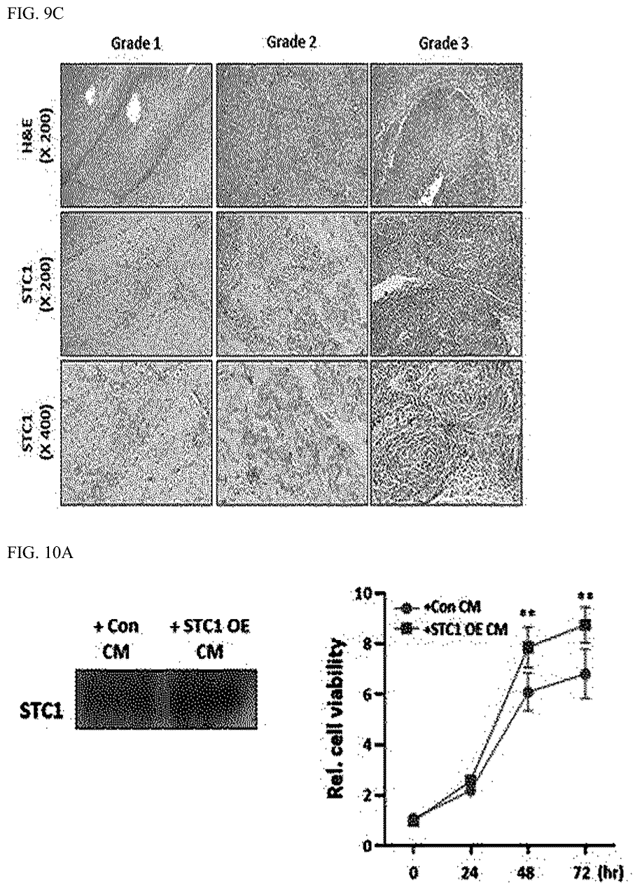

Then, in order to determine whether STC1 regulated lung metastasis, an overall experimental schematic diagram was as follows by injecting control and STC1-overexpressing stable cells into the mouse tail vein (FIG. 9A). It was observed that changes in body weight and lung metastasis between the two groups increased over time in both STC1-overexpressing stable cell mouse groups compared to the control mouse group (FIG. 9A). More and larger lung nodules were identified in the mouse group injected with the STC1 overexpressing stable cell line compared to the control cell mouse group (FIG. 9B). Higher expression of Ki67 and STC1 was confirmed in the lung tissue of mice injected with STC1-overexpressing stable cells (FIG. 9B). In addition, STC1 protein expression was divided into Grades 1, 2, and 3 and confirmed in tissue samples of bladder cancer patients, and as the grade increased, the expression of STC1 also increased (FIG. 9C). These results suggested that STC1 played an important role in promoting tumorigenesis and lung metastasis.

### 7. Confirmation of Induction of Cell Growth, Invasion, and Migration of Cancer Cells by Secretory STC1 Confirmed in Conditioned Media (CM) of STC1-Overexpressing Stable Cells

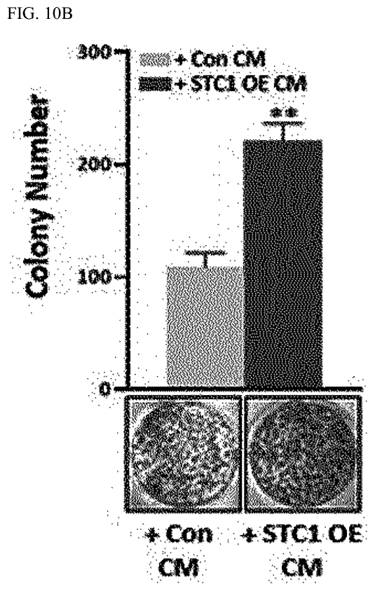

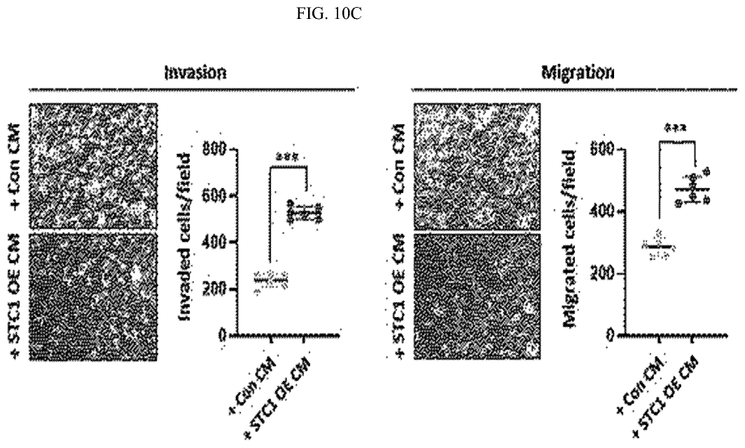

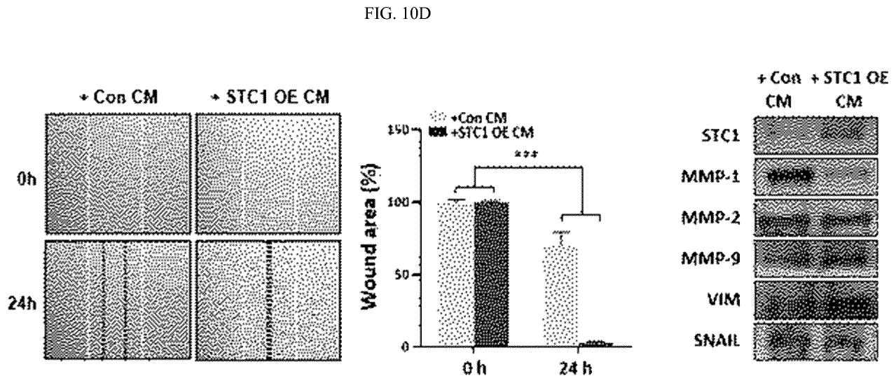

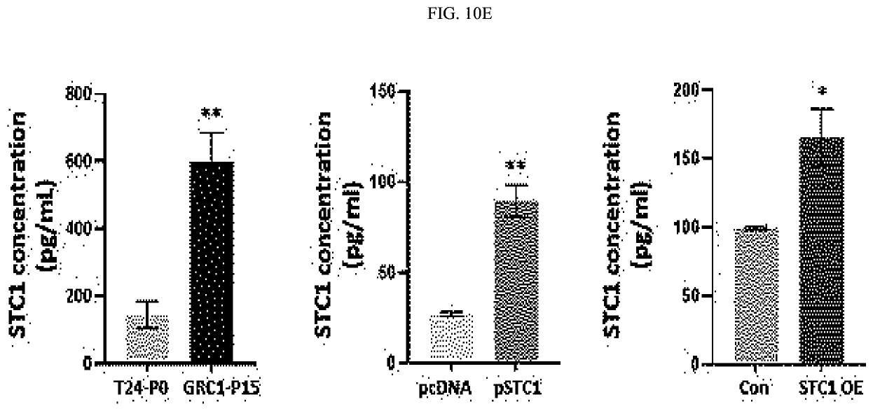

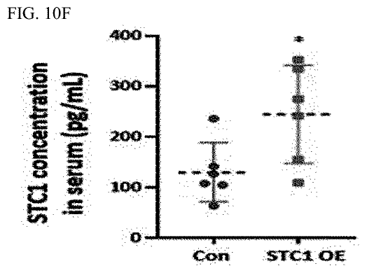

It was confirmed that the amount of secreted STC1 protein was greater in the conditioned medium of STC1 overexpressing stable cells than the conditioned medium of control cells (FIG. 10A). Cell proliferation and colony formation assays were performed for functional effects, and it was confirmed that when bladder cancer cells were treated with the conditioned medium of STC1-overexpressing stable cells, the proliferation and colony formation abilities were significantly accelerated (FIGS. 10A and 10B). In addition, in cell invasion and migration assays, bladder cancer cells cultured in the conditioned medium of the STC1-overexpressing stable cells showed higher cell migration and invasion abilities (FIG. 10C). In addition, the wound healing ability was also increased, and in Western blotting results confirmed by concentrating the conditioned medium of the STC1-overexpressing stable cells, the protein level of MMP1 was decreased, but the expression of MMP2 and 9 was increased, which suggested that secreted STC1 protein promoted EMT-related gene expression in bladder cancer cells (FIG. 10D). In addition, the amount of STC1 secreted in the conditioned medium of P15 cells was more increased than that in the conditioned medium of P0 cells (FIG. 10E). In addition, the amount of secretory STC1 protein was further increased in the conditioned media of cells transfected with the STC1 overexpressing vector and STC1 overexpressing stable cells (FIG. 10E). FIG. 10F illustrates results of performing a human STC1 ELISA on the serum by collecting mouse blood from the experiment conducted in FIG. 9 and isolating serum, and more secretory STC1 protein was found in the serum of the mouse group injected with STC1-overexpressing stable cells than the mouse group injected with control cells. In order to determine whether the STC1 secreted protein was detected in bladder cancer cells, an ELISA of human STC1 protein was performed on conditioned media of P0 and P15 cells. That is, the secreted STC1 promoted the growth, invasion, and migration of bladder cancer cells.

### 8. Confirmation of Possibility of Diagnosing and Predicting Prognosis of Bladder Cancer Patients Through Secretory STC1 Protein Identified in Serum and Urine of Bladder Cancer Patients

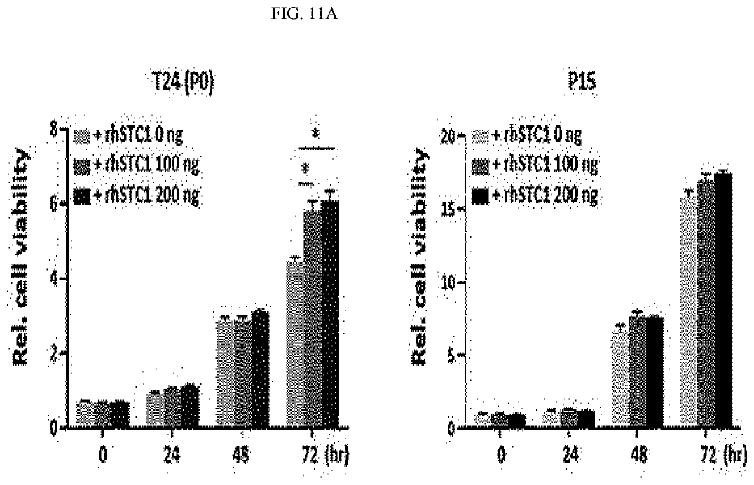

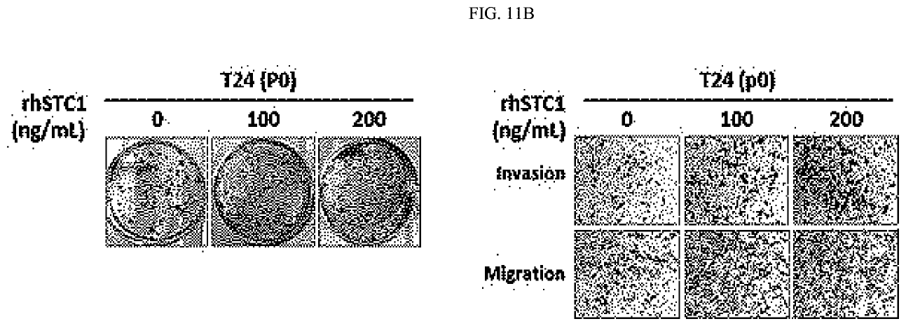

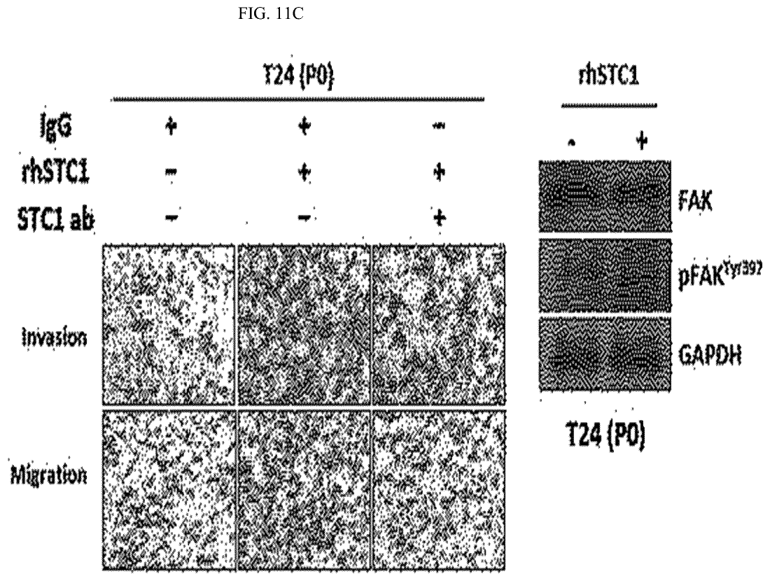

In the present invention, it was confirmed that the secreted STC1 protein was related to the metastasis of bladder cancer. To identify the STC1 protein secreted in the conditioned medium of bladder cancer cells and confirm the effect, P0 and P15 cells were treated with 0, 100, and 200 ng/ml of recombinant human STC1 (rhSTC1) protein for 0, 24, 48, and 72 hours, respectively. Cell viability was increased by rhSTC1 treatment in P0 cells at 72 hours. However, there was no effect on P15 cells (FIG. 11A). The cell proliferation ability confirmed by treating bladder cancer cells with rhSTC1 was increased, and the colony formation ability and cell migration and invasion ability in P0 cells treated with 100 and 200 ng/mL rhSTC1 were also increased (FIG. 11B). Thereafter, it was confirmed that the metastatic effect of rhSTC1 was lost in bladder cancer cells by blocking rhSTC1 with STC1 antibody (FIG. 11C). After treating the cells with rhSTC1, the expression of phosphorylated-FAK (p-FAK) was increased (FIG. 11C).

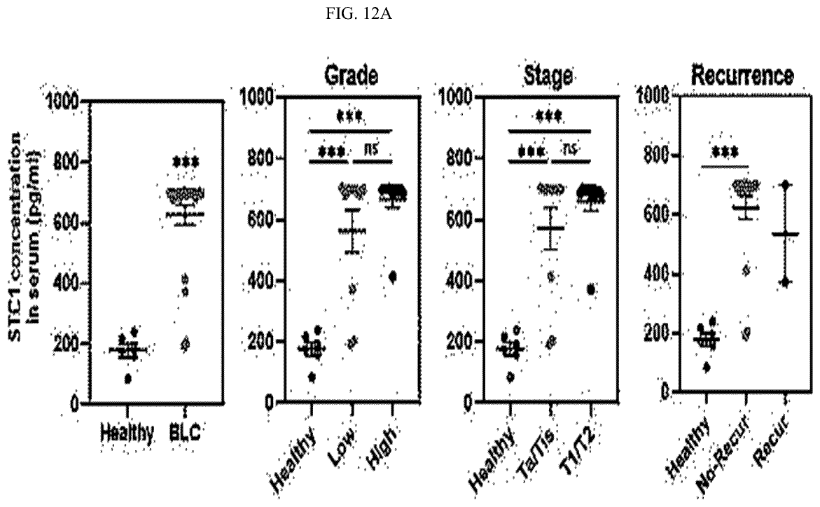

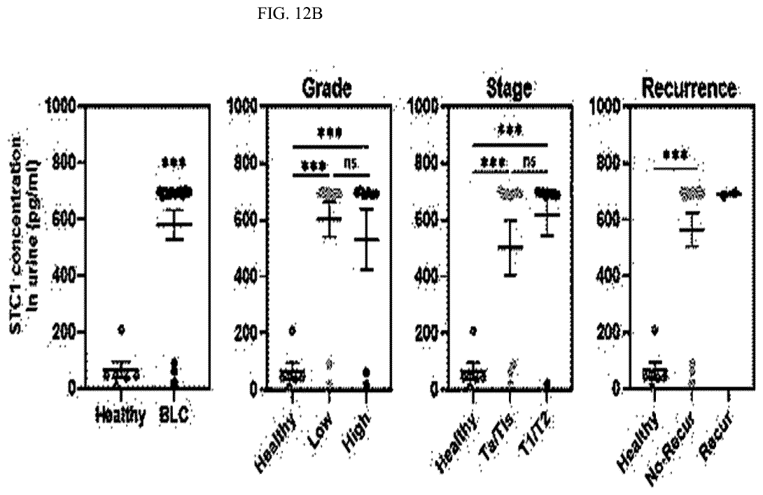

In order to confirm the effectiveness of STC1 as a biomarker for diagnosing and predicting the prognosis of bladder cancer patients, the expression of STC1 was confirmed in the serum and urine of healthy control patients and bladder cancer patients (FIG. 12). The increased level of secretory STC1 was detected in the serum of bladder cancer patients compared to the healthy control patients (FIG. 12A). Detection of secreted STC1 was also confirmed in the urine of bladder cancer patients compared to control patients, and a tendency to vary depending on the patient's grade and T stage was confirmed. Samples with identified recurrence showed no differences (FIG. 12B). These results suggest that bladder cancer may be identified using the STC1 proteins secreted from the urine and serum of bladder cancer patients. These results confirmed that STC1 may be used as a biomarker candidate.

Accordingly, STC1, a novel biomarker for diagnosing or predicting the prognosis of cancer according to the present invention, was found to be related to poor prognosis of cancer patients according to the expression level, and was found to be overexpressed in various cancer cell lines. In addition, STC1 was found to be a biomarker related to the proliferation, invasion, and migration (metastasis) of cancer cells. In addition, it was found that STC1 is detected in the serum or urine of bladder cancer patients, and may be effectively used for diagnosing and predicting the prognosis of bladder cancer by identifying differences in expression according to the patient's clinical stage.

Hereinabove, the present invention has been described with reference to preferred examples thereof. It will be understood to those skilled in the art that the present invention may be implemented as modified forms without departing from an essential characteristic of the present invention. Therefore, the disclosed examples should be considered in an illustrative viewpoint rather than a restrictive viewpoint. The scope of the present invention is illustrated by the appended claims rather than by the foregoing description, and all differences within the scope of equivalents thereof should be construed as being included in the present invention.

1. A novel Stanniocalcin-1 (STC1) biomarker for diagnosing or predicting the metastasis or prognosis of cancer.

2. A biomarker composition for diagnosing or predicting the metastasis or prognosis of cancer, comprising an agent capable of measuring the expression level of Stanniocalcin-1 (STC1) of claim 1.

3. The composition of claim 2, wherein the STC1 comprises a base sequence represented by SEQ ID NO: 1.

4. The composition of claim 2, wherein the cancer is selected from the group consisting of bladder cancer, breast cancer, glioblastoma, prostate cancer, cerebrospinal tumor, head and neck cancer, lung cancer, thymoma, mesothelioma, esophageal cancer, gastric cancer, colon cancer, liver cancer, pancreas cancer, biliary tract cancer, kidney cancer, testicular cancer, germ cell tumor, ovarian cancer, cervical cancer, endometrial cancer, lymphoma, acute leukemia, chronic leukemia, multiple myeloma, sarcoma, malignant melanoma and skin cancer.

5. The composition of claim 2, wherein when the expression of STC1 is increased compared to a reference value of a control group, the growth, invasion, or migration of cancer cells is increased.

6. The composition of claim 2, wherein when the expression of STC1 is increased compared to the reference value of the control group, the clinical stage of cancer is increased.

7. The composition of claim 2, wherein the STC1 is measured in a sample isolated from a subject.

8. The composition of claim 7, wherein the sample is selected from the group consisting of tissue, cells, whole blood, serum, plasma, saliva, sputum, cerebrospinal fluid, and urine.

9. A kit for diagnosing or predicting the metastasis or prognosis of cancer comprising the composition of claim 1.

10. An information providing method for diagnosing or predicting the metastasis or prognosis of cancer comprising:
isolating a biological sample from a subject;
measuring the expression level of Stanniocalcin-1 (STC1) of claim 1 in the isolated biological sample; and
comparing the expression level of STC1 with a reference value of a control group.

11. The method of claim 10, wherein the biological sample is selected from the group consisting of tissue, cells, whole blood, serum, plasma, saliva, sputum, cerebrospinal fluid, and urine.

12. The method of claim 10, wherein when the expression of STC1 is increased compared to the reference value of the control group, it is determined as cancer.

13. The method of claim 12, wherein the cancer is selected from the group consisting of bladder cancer, breast cancer, glioblastoma, prostate cancer, cerebrospinal tumor, head and neck cancer, lung cancer, thymoma, mesothelioma, esophageal cancer, gastric cancer, colon cancer, liver cancer, pancreas cancer, biliary tract cancer, kidney cancer, testicular cancer, germ cell tumor, ovarian cancer, cervical cancer, endometrial cancer, lymphoma, acute leukemia, chronic leukemia, multiple myeloma, sarcoma, malignant melanoma and skin cancer.

14. The method of claim 10, wherein when the expression of STC1 is increased compared to the reference value of the control group, it is determined that the growth, invasion, or migration of cancer cells is increased.

15. The method of claim 10, wherein when the expression of STC1 is increased compared to the reference value of the control group, it is determined that the clinical stage of cancer is increased.

16. A screening method of an anticancer agent comprising:
isolating a biological sample from a subject;
treating the isolated biological sample with a candidate substance;
measuring the expression level of Stanniocalcin-1 (STC1) of claim 1 in the biological sample treated with the candidate substance; and
comparing the expression level of STC1 with a reference value of a control group.

17. The method of claim 16, wherein when the expression of STC1 is low compared to the reference value of the control group, it is determined to have an anticancer effect.

18. A method for diagnosing or predicting the metastasis or prognosis of cancer comprising:
isolating a biological sample from a subject;
measuring the expression level of Stanniocalcin-1 (STC1) of claim 1 in the isolated biological sample; and
comparing the expression level of STC1 with a reference value of a control group.
